SEQUENCE LISTING (76 sequences)

SEQ ID NO: 1 - protein, 263 aa, Homo sapiens
MFRNQYDNDVTVWSPQGRIHQIEYAMEAVKQGSATVGLKSKTHAVLVALKRAQSELAAHQKKILHVDNHIGISIAGITADARILCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 2 - protein, 239 aa, Homo sapiens
  misc_feature (3)..(3): Xaa can be any naturally occurring amino acid
MAXPCGVRLSGEARKQVEVFRQNLFQEAEEFLYRFLPQKIIYLNQLLQEDSLNVADLTSLRAPLDIPIPDPPPRDDEMETDKQEKKEVHKCGFLPGNEKVLSLLAINKPEVWTLKEKCILVITWIQHLIPKIEDGNDFGVAIQEKVLERVNAVKTKVEAFQTTISKYFSERGDAVAKASKETHVMDYRALVHERDEAAYGELRAMVLDLRAFYAELYHIISSNLEKIVNPKGEEKPSMY

SEQ ID NO: 3 - protein, 8 aa, Homo sapiens
MPCAELVR

SEQ ID NO: 4 - protein, 12 aa, Homo sapiens
LEGEACGVYTPR

SEQ ID NO: 5 - protein, 13 aa, Homo sapiens
TPCQQELDQVLER

SEQ ID NO: 6 - protein, 7 aa, Homo sapiens
HGLYNLK

SEQ ID NO: 7 - protein, 18 aa, Homo sapiens
EGSCPQVNINFPQLGLCR

SEQ ID NO: 8 - protein, 14 aa, Homo sapiens
DQCQVDSQCPGQMK

SEQ ID NO: 9 - protein, 8 aa, Homo sapiens
VSCVTPNF

SEQ ID NO: 10 - protein, 16 aa, Homo sapiens
ENQLEVLEVSWLHGLK

SEQ ID NO: 11 - protein, 11 aa, Homo sapiens
ALGHLDLSGNR

SEQ ID NO: 12 - protein, 10 aa, Homo sapiens
DLLLPQPDLR

SEQ ID NO: 13 - protein, 10 aa, Homo sapiens
VAAGAFQGLR

SEQ ID NO: 14 - protein, 13 aa, Homo sapiens
SAGDVDTLAFDGR

SEQ ID NO: 15 - protein, 12 aa, Homo sapiens
ALQSNHFELSLR

SEQ ID NO: 16 - protein, 11 aa, Homo sapiens
AYGTGFVGCLR

SEQ ID NO: 17 - protein, 13 aa, Homo sapiens
QNLFQEAEEFLYR

SEQ ID NO: 18 - protein, 9 aa, Homo sapiens
CGFLPGNEK

SEQ ID NO: 19 - protein, 10 aa, Homo sapiens
VEAFQTTISK

SEQ ID NO: 20 - protein, 9 aa, Homo sapiens
DEAAYGELR

SEQ ID NO: 21 - protein, 16 aa, Homo sapiens
AFYAELYHIISSNLEK

SEQ ID NO: 22 - protein, 13 aa, Homo sapiens
HVFGESDELIGQK

SEQ ID NO: 23 - protein, 7 aa, Homo sapiens
VVFEQTK

SEQ ID NO: 24 - protein, 15 aa, Homo sapiens
VVLAYEPVWAIGTGK

SEQ ID NO: 25 - protein, 12 aa, Homo sapiens
SNVSDAVAQSTR

SEQ ID NO: 26 - protein, 10 aa, Homo sapiens
GAEIVADTFR

SEQ ID NO: 27 - protein, 12 aa, Homo sapiens
TVVAGSSDAAQK

SEQ ID NO: 28 - protein, 16 aa, Homo sapiens
LSDVTLVPVSCSELEK

SEQ ID NO: 29 - protein, 8 aa, Homo sapiens
EVAGLWIK

SEQ ID NO: 30 - protein, 11 aa, Homo sapiens
TYGLPCHCPFK

SEQ ID NO: 31 - protein, 10 aa, Homo sapiens
IESVLSSSGK

SEQ ID NO: 32 - protein, 14 aa, Homo sapiens
AVTEQGHELSNEER

SEQ ID NO: 33 - protein, 8 aa, Homo sapiens
VISSIEQK

SEQ ID NO: 34 - protein, 11 aa, Homo sapiens
YLSEVASGDNK

SEQ ID NO: 35 - protein, 14 aa, Homo sapiens
AVTELNEPLSNEDR

SEQ ID NO: 36 - protein, 15 aa, Homo sapiens
ELETVCNDVLSLLDK

SEQ ID NO: 37 - protein, 10 aa, Homo sapiens
NCNDFQYESK

SEQ ID NO: 38 - protein, 10 aa, Homo sapiens
YLAEVASGEK

SEQ ID NO: 39 - protein, 12 aa, Homo sapiens
NSVVEASEAAYK

SEQ ID NO: 40 - protein, 15 aa, Homo sapiens
NQYDNDVTVWSPQGR

SEQ ID NO: 41 - protein, 12 aa, Homo sapiens
IHQIEYAMEAVK

SEQ ID NO: 42 - protein, 12 aa, Homo sapiens
IHQIEYAMEAVK

SEQ ID NO: 43 - protein, 12 aa, Homo sapiens
ETLPAEQDLTTK

SEQ ID NO: 44 - protein, 10 aa, Homo sapiens
LSEGFSIHTR

SEQ ID NO: 45 - protein, 14 aa, Homo sapiens
GAVYSFDPVGSYQR

SEQ ID NO: 46 - protein, 9 aa, Homo sapiens
DVFISAAER

SEQ ID NO: 47 - protein, 9 aa, Homo sapiens
DVYTGDALR

SEQ ID NO: 48 - protein, 10 aa, Homo sapiens
VAASNIVQMK

SEQ ID NO: 49 - protein, 8 aa, Homo sapiens
YYTPTISR

SEQ ID NO: 50 - protein, 11 aa, Homo sapiens
FILNLPTFSVR

SEQ ID NO: 51 - protein, 20 aa, Homo sapiens
FEGGVVIAADMLGSYGSLAR

SEQ ID NO: 52 - protein, 8 aa, Homo sapiens
AIHSWLTR

SEQ ID NO: 53 - protein, 10 aa, Homo sapiens
QPVLSQTEAR

SEQ ID NO: 54 - protein, 9 aa, Homo sapiens
FQIATVTEK

SEQ ID NO: 55 - protein, 11 aa, Homo sapiens
NVNQSLLELHK

SEQ ID NO: 56 - protein, 10 aa, Homo sapiens
ELGDHVTNLR

SEQ ID NO: 57 - protein, 13 aa, Homo sapiens
DDVALEGVSHFFR

SEQ ID NO: 58 - protein, 7 aa, Homo sapiens
ALFQDIK

SEQ ID NO: 59 - protein, 15 aa, Homo sapiens
LNQALLDLHALGSAR

SEQ ID NO: 60 - protein, 15 aa, Homo sapiens
LGGPEAGLGEYLFER

SEQ ID NO: 61 - protein, 20 aa, Homo sapiens
MHITLCDFIVPWDTLSTTQK

SEQ ID NO: 62 - protein, 8 aa, Homo sapiens
YQMGCECK

SEQ ID NO: 63 - protein, 9 aa, Homo sapiens
SDGSCAWYR

SEQ ID NO: 64 - protein, 17 aa, Homo sapiens
EFFPIADGDQQSPIEIK

SEQ ID NO: 65 - protein, 9 aa, Homo sapiens
GGPLTGSYR

SEQ ID NO: 66 - protein, 9 aa, Homo sapiens
ITDTLDSIK

SEQ ID NO: 67 - protein, 10 aa, Homo sapiens
ATNDSVVADK

SEQ ID NO: 68 - protein, 7 aa, Homo sapiens
IHFIAPK

SEQ ID NO: 69 - protein, 20 aa, Homo sapiens
LPFTALGSGQDAALAVLEDR

SEQ ID NO: 70 - protein, 10 aa, Homo sapiens
TLSSPTEPVK

SEQ ID NO: 71 - protein, 7 aa, Homo sapiens
EIVNSLK

SEQ ID NO: 72 - protein, 11 aa, Homo sapiens
LQADDNGDPGR

SEQ ID NO: 73 - protein, 8 aa, Homo sapiens
ELESEVNK

SEQ ID NO: 74 - protein, 15 aa, Homo sapiens
ALASECAQHLSLPLR

SEQ ID NO: 75 - protein, 8 aa, Homo sapiens
VPTPQAIR

SEQ ID NO: 76 - protein, 16 aa, Homo sapiens
AAQGLLACGVAQGALR

(19) United States
(12) Patent Application Publication
(10) Pub. No.: US 2014/0121127 A1
(43) Pub. Date: May 1, 2014

(54) Methods and Compositions for Diagnosis of Ovarian Cancer

(71) Applicant: THE WISTAR INSTITUTE OF ANATOMY AND BIOLOGY, PHILADELPHIA, PA (US)

(72) Inventors: David W. Speicher, Berwyn, PA (US); Hsin Yao Tang, Media, PA (US); Lynn A. Beer, Medford, NJ (US)

(73) Assignee: THE WISTAR INSTITUTE OF ANATOMY AND BIOLOGY, PHILADELPHIA, PA (US)

(21) Appl. No.: 14/065,923

(22) Filed: Oct. 29, 2013

Related U.S. Application Data
(60) Provisional application No. 61/720,616, filed on Oct. 31, 2012.

(51) Int. Cl. G01N 33/68

(52) U.S. Cl. CPC G01N 33/6893
USPC 506/9; 530/389.8; 506/18; 530/387.3

(57) ABSTRACT

Methods and compositions are provided for diagnosing ovarian cancer in a mammalian subject, preferably in a serum or plasma sample of a human subject. The methods and compositions enable the detection or measurement in the sample or from a protein level profile generated from the sample, the protein level of one or more specified biomarkers. Comparing the protein level(s) of the biomarker(s) in the subject's sample or from protein abundance profile of multiple biomarkers, with the level of the same biomarker(s) or profile in a reference standard, permits the determination of a diagnosis of ovarian cancer, or the identification of a risk of developing ovarian cancer, or enables the monitoring of the status of progression or remission of ovarian cancer in the subject followed during a therapeutic protocol.

### CROSS-REFERENCE TO RELATED APPLICATIONS

This application claims the benefit of the priority of U.S. Provisional Patent Application No. 61/720,616, filed Oct. 31, 2012, which application is incorporated herein by reference in its entirety.

### STATEMENT REGARDING FEDERALLY SPONSORED RESEARCH OR DEVELOPMENT

This invention was made with government support under Grant Nos. CA131582 and CA10815 awarded by the National Institutes of Health. The government has certain rights in this invention.

### BACKGROUND OF THE INVENTION

Epithelial ovarian cancer (EOC) is the fifth-leading cause of cancer-related deaths in women, with a higher fatality-to-case ratio than any other gynecologic malignancy in the United States.¹⁻³ A major challenge in EOC is that greater than two-thirds of cases are diagnosed at advanced stages (Stages 3 or 4), when five-year survival is about 33%. In contrast, when the disease is diagnosed at Stage 1, five-year survival is approximately 90%.² CA125 is the biomarker most commonly used to detect and monitor EOC. However, 50-60% of early-stage EOC does not express CA125. In addition, while greater than 80% of advanced EOC has elevated CA125, this is not a sufficient diagnosis, as CA125 levels are also elevated in a number of other cancerous conditions.^{1, 4-6} Due to the low incidence of ovarian cancer in the general population, the specificity and sensitivity requirements for early screening are quite high, and to achieve suitable performance is likely to require a panel of biomarkers superior to most existing biomarkers.^{2, 7-9} Additional biomarkers, either instead of or in conjunction with CA125, are needed for predicting clinical outcome, stratifying therapeutic options, monitoring response to therapy, and detecting reoccurrence of the disease.

Although proteomic technologies have improved dramatically, discovering novel blood biomarkers for cancers remains formidable due to the vast complexity of the plasma proteome and the likelihood that any tumor-specific proteins will be present at very low abundance. In addition, comparison of patient and control serum or plasma is complicated by the fact that EOC, as well as other cancers and many other conditions, induce an acute-phase reaction or inflammatory response that is not specific to a single disease.¹⁰ These changes affect levels of a substantial number of high- and medium-abundant plasma proteins. Identifying cancer-specific changes in the context of this great complexity and inflammation-induced variability is very difficult when patient serum samples are directly analyzed to discover new biomarkers using proteomics. For this reason, many investigators have turned to alternative strategies for initial discovery of candidate biomarkers, including proteome analysis of: EOC cell lines, cell surface proteins in EOC cell lines, proteins shed by these cells into the media (the secretome), and patient ascities.¹¹⁻¹⁶ While all of these methods identify many proteins associated with EOC, a critical missing factor is that it is not apparent which of these proteins will migrate into the blood and be potential EOC serum biomarkers. Furthermore, changes in abundance levels of a protein in the tumor or shed by the tumor into the interstitial space do not necessarily correlate with their abundance levels in the blood.

### SUMMARY OF THE INVENTION

In one aspect, the invention provides a diagnostic reagent comprising at least one ligand capable of specifically complexing with, binding to, or quantitatively detecting or identifying a single target biomarker, wherein at least one ligand is associated with a detectable label or with a substrate. The biomarker is selected from agrin and agrin fragments (AGRN); proteasome activator complex subunit 2 (PSME2); triosephosphate isomerase (TPI1); N(G),N(G)-dimethylarginine dimethylaminohydrolase 2 (DDAH2); GM2 ganglioside activator protein (GM2A); 14-3-3 protein beta/alpha (YWHAB); 14-3-3 protein eta (YWHAH); proteasome subunit alpha type-1 (PSMA1); proteasome subunit beta type-1 (PSMB1); proteasome subunit beta type-2 (PSMB2); proteasome subunit beta type-4 (PSMB4); ferritin heavy chain (FTH1); ferritin light chain (FTL); metalloproteinase inhibitor 2 (TIMP2); carbonic anhydrase 13 (CA13); proteasome subunit beta type-10 (PSMB10); fibroleukin (FGL2); peptidoglycan recognition protein 1 (PGLYRP1); and an isoform, pro-form, modified molecular form, or peptide fragment of any of the above biomarkers, proteins in the same biomarker family or expressed from a related gene, and proteins having at least 20%, at least 30%, at least 40%, at least 50%, at least 60%, at least 70%, at least 80%, at least 90%%, at least 95% or at least 99% sequence homology or sequence identity with any of the above biomarkers. In certain embodiments, the biomarker sequence or ligand in the reagent or device is associated with a molecule or moiety capable alone or in combination with one or more additional molecules of generating a detectable signal. In other embodiments, the biomarker sequence or ligand in the reagent or device is associated with a substrate on which the sequence or ligand is immobilized. In one embodiment, the sample is serum. In another embodiment, the sample is plasma.

In another aspect, a diagnostic reagent or device comprises a ligand capable of specifically complexing with, binding to, or quantitatively detecting or identifying the biomarker proteasome subunit beta type-4 (PSMB4) or an isoform, pro-form, modified molecular form, or unique peptide fragment or nucleic acid fragment thereof.

In another aspect, the diagnostic reagent or device comprises a set of multiple biomarkers or multiple ligands to biomarkers, each ligand individually capable of specifically complexing with, binding to, or quantitatively detecting or identifying a single biomarker. In one embodiment of such diagnostic reagents or devices, one required biomarker is PSMB4. In another embodiment, at least one biomarker is known to be associated with ovarian cancer. In one embodiment, a required biomarker is CA125, or an isoform, pro-form, modified molecular form, or peptide fragment thereof. In another embodiment, a required biomarker is selected from cancer antigen 125 (CA125), chloride intracellular channel protein 1 (CLIC1), peroxiredoxin-6 (PRDX6), cathepsin-D (CTSD, including CTSD-30K or CTSD-52K), insulin-like growth factor binding protein 2 (IGFBP2), WAP four-disulfide core domain protein 2 (WFDC2, also called HE4), leucine rich alpha-2-glycoprotein 1(LRG1), and an isoform, pro-form, modified molecular form, or peptide fragment thereof.

In another aspect, the invention provides a kit, panel or microarray comprising at least two diagnostic reagents described herein, each reagent identifying a different biomarker. In one embodiment, the kit comprises diagnostic reagents that bind to or complex individually with 2, 3, 4, 5, 6, 7, 8, 9, 10, 11, 12, 13, 14, 15, 16, 17, 18 or more biomarkers. In another embodiment, the kit, panel or microarray includes diagnostic reagents that bind to or complex individually with at least one additional known marker, isoform, pro-form, modified molecular form, or peptide fragment or homolog thereof.

In a further aspect, a method for diagnosing or detecting or monitoring the progress of ovarian cancer in a subject is provided. In one embodiment, the method comprises contacting a sample obtained from a test subject with a diagnostic reagent or device comprising a ligand capable of specifically complexing with, binding to, or quantitatively detecting or identifying at least one biomarker selected from agrin and agrin fragments (AGRN); proteasome activator complex subunit 2 (PSME2); triosephosphate isomerase (TPI1); N(G),N(G)-dimethylarginine dimethylaminohydrolase 2 (DDAH2); GM2 ganglioside activator protein (GM2A); 14-3-3 protein beta/alpha (YWHAB); 14-3-3 protein eta (YWHAH); proteasome subunit alpha type-1 (PSMA1); proteasome subunit beta type-1 (PSMB1); proteasome subunit beta type-2 (PSMB2); proteasome subunit beta type-4 (PSMB4); ferritin heavy chain (FTH1); ferritin light chain (FTL); metalloproteinase inhibitor 2 (TIMP2); carbonic anhydrase 13 (CA13); proteasome subunit beta type-10 (PSMB10); fibroleukin (FGL2); peptidoglycan recognition protein 1 (PGLYRP1); and an isoform, pro-form, modified molecular form, or peptide fragment of any of the above biomarkers, proteins in the same biomarker family or expressed from a related gene, and proteins having at least 20%, at least 30%, at least 40%, at least 50%, at least 60%, at least 70%, at least 80%, at least 90%%, at least 95% or at least 99% sequence homology or sequence identity with any of the above biomarkers. The protein levels of the selected biomarker(s) are then detected or measured in the sample or from a protein level profile generated from the sample. The protein levels of the biomarker(s) (or peptides of biomarker serving as surrogates of the protein, e.g., those discussed in FIG. 7) in the subject's sample are compared with the level of the same biomarker in a reference standard. A significant change in protein level of the subject's sample from that in the reference standard indicates a diagnosis, risk, or the status of progression or remission of ovarian cancer in the subject. In one embodiment of this method, an additional step involves detecting or measuring in the sample or from a protein level profile generated from the sample, the protein levels of one or more additional known ovarian cancer biomarkers; and comparing the protein levels of the known biomarker in relation to the levels of the additional biomarkers in the subject's sample with the same biomarkers in a reference standard or profile.

In a further aspect, a method for diagnosing or detecting or monitoring the progress of ovarian cancer in a subject is provided. In one embodiment, the method comprises contacting a sample obtained from a test subject with a diagnostic reagent or device comprising a ligand capable of specifically complexing with, binding to, or quantitatively detecting or identifying PSMB4 or an isoform, pro-form, modified molecular form, or unique peptide fragment or nucleic acid fragment thereof. The protein levels of PSMB4 (or peptides of PSMB4 serving as surrogates of the PSMB4 protein, e.g., those discussed in FIG. 7) are then detected or measured in the sample or from a protein level profile generated from the sample. The protein levels of the PSMB4 biomarker in the subject's sample are compared with the level of the same biomarker in a reference standard. A significant change in protein level of the subject's sample from that in the reference standard indicates a diagnosis, risk, or the status of progression or remission of ovarian cancer in the subject. Analogous methods are provided for other biomarkers or biomarker sets described herein.

In another aspect, use of any of the diagnostic reagents described herein in a method for the diagnosis of ovarian cancer is provided.

Other aspects and advantages of these compositions and methods are described further in the following detailed description of the preferred embodiments thereof.

### BRIEF DESCRIPTION OF THE DRAWINGS

FIG. 1. Scheme for comparison of xenograft plasma and tumor secretome for ovarian cancer biomarker discovery.

FIG. 2. Comparison of proteins identified in xenograft secretome and plasma. (A) The numbers of unique (nonredundant) proteins identified in the OVCAR-3 xenograft tumor secretome and mouse plasma. Data are shown after sorting proteins based on species classification where at least one peptide was uniquely human, uniquely mouse, or indistinguishable (Indist.), that is, all detected peptides were common to human and mouse homologs. (B) Overlap of high-confidence protein identifications in the tumor secretome and xenograft plasma. Protein counts are for proteins identified by at least two peptides. Overlap was assessed by exact matches of database accession numbers.

FIG. 3. Sequence coverage for selected human proteins from the xenograft plasma and tumor secretomes. Examples of candidate biomarkers, PSMA1 (SEQ ID NO: 1) and PSME2 (SEQ ID NO: 2), where increased sequence coverage is obtained from analysis of the tumor secretome (underlined peptides) compared to the xenograft plasma (grey highlight). Tryptic sites (K or R) are indicated in bold lettering.

FIG. 4. Quantitative comparisons of candidate biomarkers using label-free discovery mode LC-MS/MS analysis of patient serum pools. Summed protein intensities from a Rosetta Elucidator label-free analysis are shown for benign (B, n=9) and three different late-stage ovarian cancer pools (C1, n=9; C2, n=9; C3, n=5). (A) Representative proteins that failed this screen because intensities were lower or showed no difference in cancer pools compared with benign disease. (B) Representative proteins selected for further validation because they showed elevated levels in all cancer pools compared with benign sera. (C) Four different isoforms of the proteasome complex selected for further validation because they showed elevated levels in all cancer pools compared with benign sera.

FIG. 5. Gene expression of candidate biomarkers in ovarian tissues. Gene expression levels for normal ovary tissue (N; n=4) and papillary serous ovarian carcinoma primary tumor samples (C, n=14) are shown. (A) Previously reported ovarian cancer biomarkers. (B) Representative novel proteins identified in the plasma and tumor secretome. (C) Representative candidate proteasome proteins. Microarray hybridization data were processed and scaled as previously described.^{29, 40} Data were extracted from www.BioGPS.org.

FIG. 6. Preliminary MRM validation using large patient serum pools. Normalized relative protein amounts are shown for serum pools of normal (N; n=9), benign (B; n=10), early-stage ovarian cancer (E; stages 1 and 2; n=18), and late-stage ovarian cancer (L; stage 3; n=29). Ratios of late-stage cancer to benign disease and normal donors are shown above the histograms. (A) Upper panel—ovarian cancer biomarkers previously reported by others and analyzed in this study; Lower panel—average values for novel biomarkers taken from our previous report.¹⁸ (B) Novel biomarkers identified in the mouse xenograft plasma and verified, typically with greater sequence coverage in the tumor secretome. (C) Novel biomarkers identified in the tumor secretome and ranked as high-priority candidates but not detected in the mouse plasma. (D) Additional novel biomarkers identified in the label-free patient serum pool comparison.

FIG. 7. Peptide transitions monitored by MRM. FIGS. 7A-7C show the peptides and transitions used in the integrated multiplex MRM assay, as well as the resulting relative quantitative data for the four pools, for each of the biomarkers indicated in Table 2. The peptide sequences in the FIG. 7 are SEQ ID NOs: 3-76, consecutively.

FIG. 8. Sample classification for ovarian cancer patient sera. FIGS. 8A and 8B show the classification, histotype, and pool designation for serum samples derived from benign patients advanced ovarian cancer patients. For the LCMS, benign samples were combined in a single pool (pool B, n=9), and advanced cancer specimens were combined in three pools (pool C1: stage 3, n=9; pool C2: stage 3, n=9; pool C3: stage 4, n=5). For the MRM studies, a normal serum pool (pool N; n=9), a benign ovarian tumor pool (pool B, n=10), an early-stage ovarian cancer pool (pool E: stage 1 and 2, n=18), and a late-stage cancer pool (pool L: stage 3, n=29) were prepared.

### DETAILED DESCRIPTION OF THE INVENTION

The compositions and methods described herein provide means for diagnosing or detecting the existence or absence of, or monitoring the progress of, ovarian cancer in a subject using one or more of the biomarkers identified in Table 1 in optional combination with one or more known ovarian cancer-associated biomarkers. As demonstrated in the examples below, the inventors performed in-depth proteome analysis of plasma from SCID mice bearing ovarian tumors formed by a serous ovarian cancer line and compared the results with a corresponding tumor secretome. Using the results from this study, the inventors identified the most promising candidate biomarkers by identifying those exhibiting elevated levels in serum pools from ovarian cancer patients using MRM studies. This strategy proved to be highly efficient, as all proteins selected for MRM showed substantial differences between cancer and controls, suggesting that all 18 novel biomarkers identified in this study are useful in detection of ovarian cancer.

In one embodiment, low-abundance human proteins are present at less than about 100 ng/ml in normal human serum. In other embodiments, low-abundance human proteins are present at less than about 80, 50, 30, 20, 10 or 1 ng/ml in normal human serum. Such difficult-to-detect proteins, which are in lower abundance in the serum, are more tumor-specific than proteins present in greater concentrations in the serum.

Because certain biomarkers identified in Table 1 were found by the inventors to be secreted in low abundance into serum from the tumor tissue, the compositions and methods described herein involve detection and/or quantitative evaluation of these biomarkers, individually or in combination with the other biomarkers in Table 1 and/or with additional known ovarian cancer biomarkers such as the previously identified biomarkers CLIC1, PRDX6, and CTSD, among others identified in PCT/US2012/54136, and CLIC4 and at least four tropomyosins biomarker proteins (TPM1 variant 6, TPM2, TPM3, and TPM4), among others identified in U.S. Provisional Patent Application No. 61/709,695, in a sample, desirably a serum or plasma or blood sample, from a subject. Other previously known EOC biomarkers include CA125, IGFBP2, WFDC2 (HE4), and LRG1. Such detection and evaluation permits diagnosis of ovarian cancer in a less invasive manner than is currently available using a tissue biopsy or a surgical sample. The methods and compositions also may be used as a diagnostic to avoid surgical diagnostic procedures. The identification and use of such a panel of biomarker reagents provides a critical, more precise basis of knowledge to incorporate into pre-clinical and clinical diagnostic assays targeting these biomarkers.

Protein abundance levels of biomarkers in blood, in some embodiments, are dependent upon expression levels in tissues of origin (e.g., ovarian tumors), as well as rate of shedding into the blood and rate of clearance from the blood. While increased expression in a tumor often will correlate with increased abundance levels being observed in the blood, this is not necessarily always true. Therefore, the methods and compositions in one aspect refer to compositions that detect protein biomarkers and to protein assay methods. However, one of skill in the art, given the teachings contained herein, would readily understand that nucleic acid expression levels of the biomarkers and reagents and methods for their detections may be similarly practiced, without undue experimentation.

Diagnostic reagents that can detect and measure the target biomarkers and sets of biomarkers identified herein and methods for evaluating the level or ratios of these target biomarkers vs. their level(s) in a variety of reference standards or controls of different conditions or stages in ovarian cancer are valuable tools in the early detection and monitoring of ovarian cancer.

In one embodiment, the compositions and methods allow the detection and measurement of the protein levels or ratios of one or more “target” biomarkers of Table 1 in a biological sample, preferably a biological fluid. Diagnostic reagents that can detect and measure these target biomarkers and methods for evaluating the level or ratios of these target biomarkers vs. their level(s) in a variety of reference standards or controls of different conditions or stages in ovarian cancer are valuable tools in the early detection and monitoring of ovarian cancer.

As described below in the examples, the utility of analyzing both xenograft mouse plasma and the corresponding tumor secretome in parallel was demonstrated. The presence of human proteins in the plasma unambiguously demonstrated these proteins were produced by the tumor and shed into the blood, but many of these assignments were based upon only a few peptides. In contrast, many, but not all, human proteins in the plasma were identified by many more peptides in the tumor secretome. This generally greater sequence coverage for proteins of interest provided additional proteotypic peptides as targets for setting up MRM assays. While identification of human proteins in mouse plasma is the most promising strategy for ovarian cancer biomarker discovery, additional candidates can be identified directly from the secretome. The challenge here is to select the best candidates from among the several thousand human proteins identified. Our pilot validation of a small number of candidates identified in the secretome, but not the plasma, demonstrates the value of further mining this large dataset of biomarker candidates. This capacity to now discover hundreds to thousands of candidate biomarkers requires better strategies for early-stage verification and validation of candidate biomarkers so that less effort is invested on validation of proteins that cannot be detected in patients or do not correlate with ovarian cancer. In this regard, an in-depth, label-free comparison of benign disease and advanced cancer patient serum pools provides an excellent verification database for prescreening candidate biomarkers prior to setting up MRM assays. By extending reverse-phase gradients for these samples to four hours, the detection sensitivity is similar to that of MRM assays using shorter gradients. That is, if a protein cannot be detected in the verification database, it will probably not be feasible to set up an MRM assay and, therefore, effort is not wasted in assay development. Furthermore, by comparing candidate biomarker levels in the benign and advanced cancer patient pools, only those proteins showing elevated levels in the cancer sera advance to MRM assay development. The value of this approach is illustrated by our ability to set up robust MRM assays for 21 of 25 proteins in the current study, while in an earlier xenograft mouse study without this verification step, our success rate in MRM assay development was only about 40%.¹⁸ Finally, applying the MRM assays to large pools of normal, benign, early, and advanced ovarian cancer identifies those biomarkers worth further validation in individual patient serum samples.

### I. DEFINITIONS

“Patient” or “subject” as used herein means a female mammalian animal, including a human, a veterinary or farm animal, a domestic animal or pet, and animals normally used for clinical research. In one embodiment, the subject of these methods and compositions is a human.

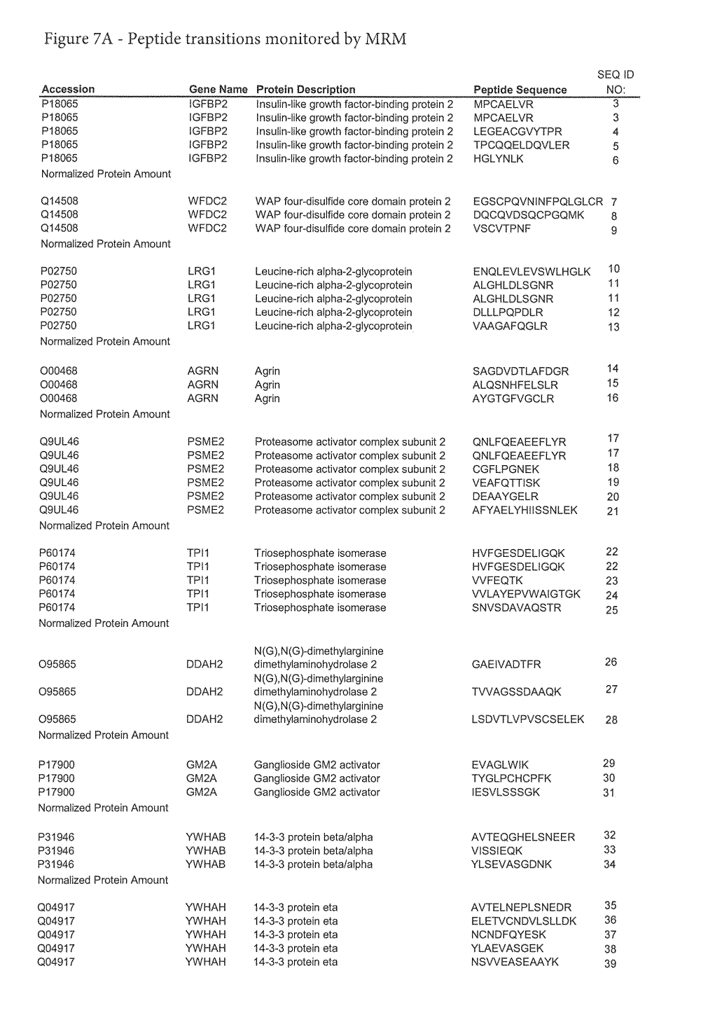

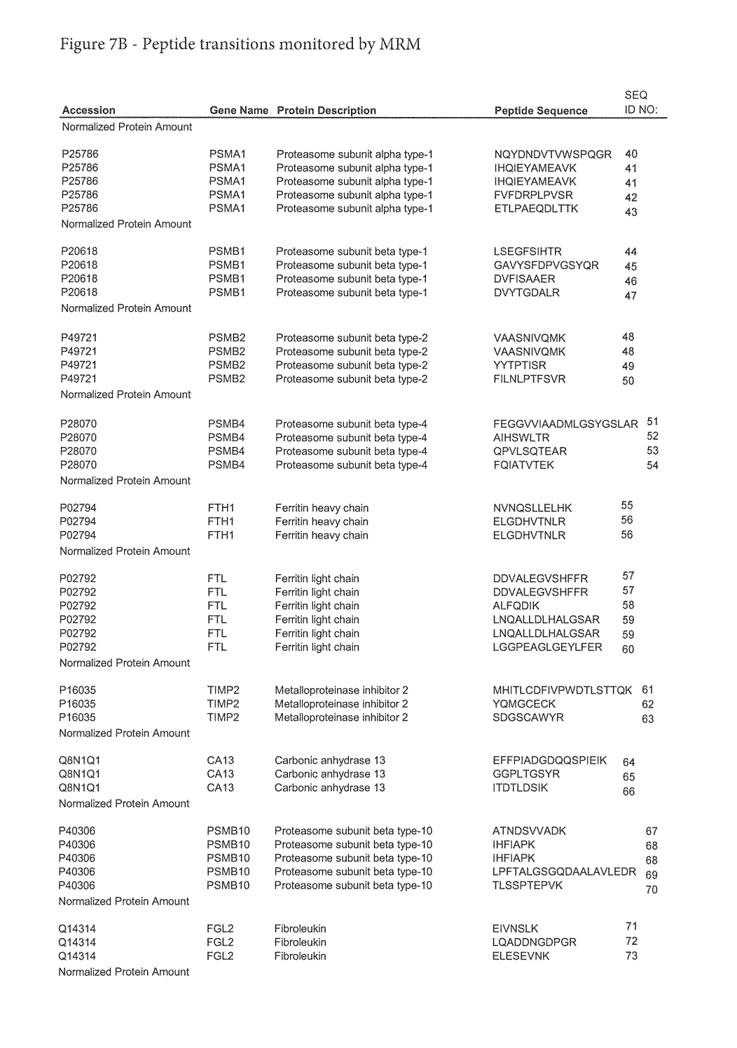

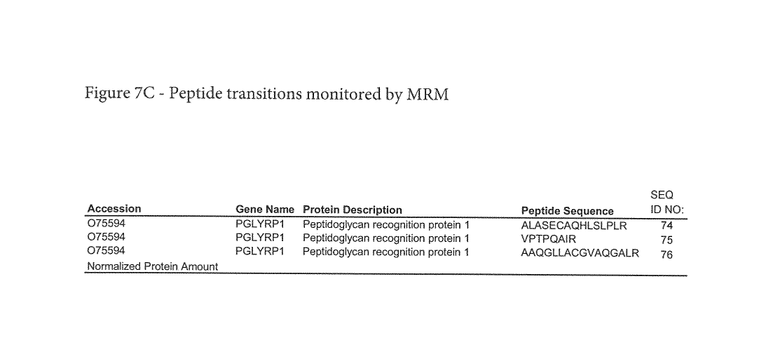

By “biomarker” or “biomarker signature” as used herein is meant a single protein or a combination of proteins or peptide fragments thereof, the protein levels or relative protein levels or ratios of which significantly change (either in an increased or decreased manner) from the level or relative levels present in a subject having one physical condition or disease or disease stage from that of a reference standard representative of another physical condition or disease stage. Throughout this specification, wherever a particular biomarker is identified by name, it should be understood that the term “biomarker” includes those listed in Table 1 below. These biomarkers may be combined to form certain sets of biomarkers or ligands to biomarkers in diagnostic reagents. Still other “additional” biomarkers are mentioned specifically herein in combination with the biomarkers of Table 1. Biomarkers described in this specification include any physiological molecular forms, or modified physiological molecular forms, isoforms, pro-forms, and peptide fragments thereof, unless otherwise specified. It is understood that all molecular forms useful in this context are physiological, e.g., naturally occurring in the species. Preferably the peptide fragments obtained from the biomarkers are unique sequences, such as those exemplified in FIG. 7. However, it is understood that fragments other than those explicitly identified may be obtained readily by one of skill in the art in view of the teachings provided herein.

TABLE 1
SELECTED BIOMARKERS OF OVARIAN CANCER

| Gene Name | Biomarker Protein Name |
|---|---|
| AGRN | Agrin and agrin fragments |
| PSME2 | Proteasome activator complex subunit 2 |
| TPI1 | Triosephosphate isomerase |
| DDAH2 | N(G),N(G)-dimethylarginine dimethylaminohydrolase 2 |
| GM2A | GM2 ganglioside activator protein (GM2A) |
| YWHAB | 14-3-3 protein beta/alpha |
| YWHAH | 14-3-3 protein eta |
| PSMA1 | Proteasome subunit alpha type-1 |
| PSMB1 | Proteasome subunit beta type-1 |
| PSMB2 | Proteasome subunit beta type-2 |
| PSMB4 | Proteasome subunit beta type-4 |
| FTH1 | Ferritin heavy chain |
| FTL | Ferritin light chain |
| TIMP2 | Metalloproteinase inhibitor 2 |
| CA13 | Carbonic anhydrase 13 |
| PSMB10 | Proteasome subunit beta type-10 |
| FGL2 | Fibroleukin |
| PGLYRP1 | Peptidoglycan recognition protein 1 |

In one embodiment, at least one biomarker of Table 1 forms a suitable biomarker signature for use in the methods and compositions. In one embodiment, at least two biomarkers form a suitable biomarker signature for use in the methods and compositions. In another embodiment, at least three biomarkers form a suitable biomarker signature for use in the methods and compositions. In another embodiment, at least four biomarkers form a suitable biomarker signature for use in the methods and compositions. In another embodiment, at least five biomarkers form a suitable biomarker signature for use in the methods and compositions. In another embodiment, at least six biomarkers form a suitable biomarker signature for use in the methods and compositions. In another embodiment, at least seven biomarkers form a suitable biomarker signature for use in the methods and compositions. In another embodiment, at least eight biomarkers form a suitable biomarker signature for use in the methods and compositions. In still further embodiments, at least 9, at least 10, at least 11, at least 12, at least 13, at least 14, at least 15, at least 16, at least 17, or all 18 of the biomarkers of Table 1 can be used alone or with additional biomarkers. Thus, from 1 to 18 of the biomarkers of Table 1, or ligands or reagents that interact with the biomarkers, can be used in diagnostic panels or arrays or kits. In another embodiment, from 1 to 18 of the biomarker/ligands of Table 1 can be used with other known biomarkers for ovarian cancer and their ligands or reagents, so that the biomarker panel or array (or diagnostic reagent or kit) contains at least 20, 30, 40, 50 or at least 60 total biomarkers (or ligands) including any numbers there between. Specific biomarker signatures can include any combination of ovarian cancer biomarkers employing at least one biomarker (or its ligand) from Table 1 and including up to all 18 biomarkers in Table 1, in any combination with another biomarker, such as CA125 or HE4. Still other biomarkers such as those identified in the references cited herein may also be present in panels with the biomarkers of Table 1.

While these biomarkers may be used individually or in various combinations as signatures, it is contemplated that multiple molecular forms or fragments of each biomarker of Table 1 and fragments/peptides as described in FIG. 7, are similarly useful in the compositions and methods described herein. For example, AGRN, is a 215 kDa protein previously identified as being upregulated in ovarian cancer tissue samples compared with normal and non-ovarian tissue samples,²⁸ but has not previously been reported to be a serum biomarker for EOC. The inventors detected by SDS-PAGE both the intact protein and as a fragment in the tumor secretome. In contrast, only the lower molecular weight fragment was detected in the xenograft plasma. Still other modified molecular forms of the biomarkers include protein modifications such as different glycosylation patterns and other conventional protein modifications of the biomarkers that occur in nature. In one embodiment, multiple molecular forms of the same protein biomarker parallel each other in patient samples. In another embodiment, one molecular form of a biomarker is a better biomarker for a condition than the other. In another embodiment, a ratio of the multiple molecular forms of the same biomarker forms a useful biomarker. Multiple molecular forms of these biomarkers can occur in blood or other biological samples. In certain embodiments, the presence of one or multiple specific molecular forms of the same biomarker, or a ratio of same, rather than overall protein levels is useful as a biomarker for a particular condition specified herein. One skilled in the art may readily reproduce the compositions and methods described herein by use of the sequences of the biomarkers, all of which are publicly available from conventional sources, such as GENBANK, UniProt or NCBI.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

By “related proteins” or “proteins of the same family” are meant expression products of different genes or related genes identified as belonging to a common family. Related proteins in the same biomarker family may or may not share related functions. Related proteins can be readily identified as having significant sequence identity either over the entire protein or a significant part of the protein that is typically referred to as a “domain”; typically proteins with at least 20% sequence homology or sequence identity can be readily identified as belonging to the same protein family.

By “homologous protein” is meant an alternative form of a related protein produced from a related gene having a percent sequence similarity or identity of greater than 20%, greater than 30%, greater than 40%, greater than 50%, greater than 60%, greater than 70%, greater than 75%, greater than 80%, greater than 85%, greater than 90%, greater than 95%, greater than 97%, or greater than 99%.

“Reference standard” as used herein refers to the source of the reference biomarker levels. The “reference standard” is preferably provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or protein level profile derived from the same biomarker or biomarkers in a reference subject or reference population. In an embodiment, in which expression of nucleic acid sequences encoding the biomarkers is desired to be evaluated, the reference standard can be an expression level of one or more biomarkers or an expression profile.

“Reference subject” or “Reference Population” defines the source of the reference standard. In one embodiment, the reference is a human subject or a population of subjects having no ovarian cancer, i.e., healthy controls or negative controls. In yet another embodiment, the reference is a human subject or population of subjects with one or more clinical indicators of ovarian cancer, but who did not develop ovarian cancer. In still another embodiment, the reference is a human subject or a population of subjects having benign ovarian nodules or cysts. In still another embodiment, the reference is a human subject or a population of subjects who had ovarian cancer, following surgical removal of an ovarian tumor. In another embodiment, the reference is a human subject or a population of subjects who had ovarian cancer and were evaluated for biomarker levels prior to surgical removal of an ovarian tumor. Similarly, in another embodiment, the reference is a human subject or a population of subjects evaluated for biomarker levels following therapeutic treatment for ovarian cancer. In still another embodiment, the reference is a human subject or a population of subjects prior to therapeutic treatment for an ovarian cancer. Similarly, in another embodiment, the reference human subject or a population of subjects without ovarian cancer but which tests positive for a protein level of CA-125 or HE4 or other known ovarian cancer biomarker. Similarly, in another embodiment, the reference human subject or a population of subjects with ovarian cancer but which tests negative for a protein level of CA125 or HE4 or other known ovarian cancer biomarker. In still other embodiments of methods described herein, the reference is obtained from the same test subject who provided a temporally earlier biological sample. That sample can be pre- or post-therapy or pre- or post-surgery.

Other potential reference standards are obtained from a reference that is a human subject or a population of subjects having early stage ovarian cancer. In another embodiment the reference is a human subject or a population of subjects having advanced stage ovarian cancer. In still another embodiment, the reference is a human subject or a population of subjects having a subtype of epithelial ovarian cancer. In still another embodiment, the reference is a human subject or a population of subjects having serous ovarian cancer or serous papillary adenocarcinoma. In still another embodiment, the reference is a human subject or a population of subjects having mucinous ovarian cancer. In still another embodiment, the reference is a human subject or a population of subjects having clear cell ovarian cancer. In still another embodiment, the reference is a subject or a population of subjects having endometrioid ovarian cancer. In another embodiment, the reference is a human subject or a population of subjects having Mullerian ovarian cancer. In another embodiment, the reference is a human subject or a population of subjects having undifferentiated ovarian cancer or an ovarian sarcoma. In another embodiment, the reference standard is a combination of two or more of the above reference standards.

Selection of the particular class of reference standards, reference population, biomarker levels or profiles depends upon the use to which the diagnostic/monitoring methods and compositions are to be put by the physician and the desired result, e.g., initial diagnosis of ovarian cancer or other ovarian condition, clinical management of patients with ovarian cancer after initial diagnosis, including, but not limited to, monitoring for reoccurrence of disease or monitoring remission or progression of the cancer and either before, during or after therapeutic or surgical intervention, selecting among therapeutic protocols for individual patients, monitoring for development of toxicity or other complications of therapy, predicting development of therapeutic resistance, and the like. Such reference standards or controls are the types that are commonly used in similar diagnostic assays for other biomarkers.

“Sample” as used herein means any biological fluid or tissue that contains the ovarian cancer biomarkers of Table 1. The most suitable samples for use in the methods and with the compositions are samples which require minimal invasion for testing, e.g., blood samples, including serum, plasma, whole blood, and circulating tumor cells. It is also anticipated that other biological fluids, such as saliva or urine, vaginal or cervical secretions, and ascites fluids or peritoneal fluid may be similarly evaluated by the methods described herein. Also, circulating tumor cells or fluids containing them are also suitable samples for evaluation in certain embodiments of this invention. The samples may include biopsy tissue, tumor tissue, surgical tissue, circulating tumor cells, or other tissue. Such samples may further be diluted with saline, buffer or a physiologically acceptable diluent. Alternatively, such samples are concentrated by conventional means. In certain embodiments, e.g., those in which expression levels of nucleic acid sequences encoding the biomarkers are desired to be evaluated, the samples may include biopsy tissue, surgical tissue, circulating tumor cells, or other tissue. In one embodiment, the sample is a tumor secretome, i.e., any fluid or medium containing the proteins secreted from the tumor. These shed proteins may be unassociated, associated with other biological molecules, or enclosed in a lipid membrane such as an exosome. In another embodiment, the sample is plasma.

By “significant change in protein level” is meant an increased protein level of a selected biomarker in comparison to that of the selected reference standard or control or relative to a predetermined cutpoint; a decreased protein level of a selected biomarker in comparison to that of the selected reference or control or relative to a predetermined cutpoint; or a combination of a pattern or relative pattern of certain increased and/or decreased biomarkers. In one embodiment, the significant changes is an increased protein level.

The degree of change in biomarker protein level can vary with each individual and is subject to variation with each population. For example, in one embodiment, a large change, e.g., 2-3 fold increase or decrease in protein levels of a small number of biomarkers, e.g., from 1 to 9 characteristic biomarkers, is statistically significant. In another embodiment, a smaller relative change in 10 or more (i.e., about 10, 20, 24, 29, or 30 or more biomarkers) is statistically significant. The degree of change in any biomarker(s) expression varies with the condition, such as type of ovarian cancer and with the size or spread of the cancer or solid tumor. The degree of change also varies with the immune response of the individual and is subject to variation with each individual. For example, in one embodiment of this invention, a change at or greater than a 1.2 fold increase or decrease in protein level of a biomarker or more than two such biomarkers, or even 3 or more biomarkers, is statistically significant. In another embodiment, a larger change, e.g., at or greater than a 1.5 fold, greater than 1.7 fold or greater than 2.0 fold increase or a decrease in expression of a biomarker(s) is statistically significant. This is particularly true for cancers without solid tumors. Still alternatively, if a single biomarker protein level is significantly increased in biological samples which normally do not contain measurable protein levels of the biomarker, such increase in a single biomarker level may alone be statistically significant. Conversely, if a single biomarker protein level is normally decreased or not significantly measurable in certain biological samples which normally do contain measurable protein levels of the biomarker, such decrease in protein level of a single biomarker may alone be statistically significant.

A change in protein level of a biomarker required for diagnosis or detection by the methods described herein refers to a biomarker whose protein level is increased or decreased in a subject having a condition or suffering from a disease, specifically ovarian cancer, relative to its expression in a reference subject or reference standard. Biomarkers may also be increased or decreased in protein level at different stages of the same disease or condition. The protein levels of specific biomarkers differ between normal subjects and subjects suffering from a disease, benign ovarian nodules, or cancer, or between various stages of the same disease. Protein levels of specific biomarkers differ between pre-surgery and post-surgery patients with ovarian cancer. Such differences in biomarker levels include both quantitative, as well as qualitative, differences in the temporal or relative protein level or abundance patterns among, for example, biological samples of normal and diseased subjects, or among biological samples which have undergone different disease events or disease stages. For the purpose of this invention, a significant change in biomarker protein levels when compared to a reference standard is considered to be present when there is a statistically significant (p<0.05) difference in biomarker protein level between the subject and reference standard or profile, or significantly different relative to a predetermined cut-point.

For example, in one embodiment, the test subject's biomarker(s) levels are compared with a healthy reference standard. If the subject has ovarian cancer, the selected EOC biomarker(s) e.g., those selected from the biomarkers of Table 1, will typically show a change in protein level from the levels in the healthy reference standard, thus permitting diagnosis of ovarian cancer. In another example, the biomarker(s) of Table 1 differentially change in protein level (either by increased or decreased protein level) when the biomarker levels or relative levels from the sample of a subject having one of the following conditions is compared to a reference subject or population having another of the following physical conditions. These “conditions” include no ovarian cancer, the presence of benign ovarian nodules, the presence of an ovarian cancer or subtype, the condition following surgical removal of an ovarian tumor; the condition prior to surgical removal of an ovarian tumor; the condition following a specific therapeutic treatment for an ovarian tumor; the condition prior to a specific therapeutic treatment for an ovarian tumor. It is further anticipated that the biomarker(s) expression levels may change and the changes may be detected during treatment for ovarian cancer. In another embodiment, a condition includes that of a subject having undiagnosed clinical symptoms of abdominal pain or other abdominal condition of unknown origin. Still other embodiments of “conditions” as defined above include early stage ovarian cancer; advanced stage ovarian cancer; a subtype of epithelial ovarian cancer; serous ovarian cancer; mucinous ovarian cancer; clear cell ovarian cancer; endometrioid ovarian cancer; Mullerian ovarian cancer; undifferentiated ovarian cancer; serous papillary adenocarcinoma; and sarcoma.

The term “ligand” refers, with regard to protein biomarkers, to a molecule that binds or complexes with a biomarker protein, molecular form or peptide, such as an antibody, antibody mimic or equivalent that binds to or complexes with a biomarker identified herein, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody” refers to an intact immunoglobulin having two light and two heavy chains or fragments thereof capable of binding to a biomarker protein or a fragment of a biomarker protein. Thus a single isolated antibody or fragment may be a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

As used herein, “labels” or “reporter molecules” are chemical or biochemical moieties useful for labeling a ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a ligand.

As used herein the term “cancer” refers to or describes the physiological condition in mammals that is typically characterized by unregulated cell growth. More specifically, as used herein, the term “cancer” means any ovarian cancer. In one embodiment, the ovarian cancer is an epithelial ovarian cancer or subtype as referred to in “conditions” above. In still an alternative embodiment, the cancer is an “early stage” (I or II) ovarian cancer. In still another embodiment, the cancer is a “late stage” (III or IV) ovarian cancer.

The term “tumor,” as used herein, refers to all neoplastic cell growth and proliferation, whether malignant or benign, and all pre-cancerous and cancerous cells and tissues.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

The term “microarray” refers to an ordered arrangement of binding/complexing array elements or ligands, e.g. antibodies, on a substrate.

In the context of the compositions and methods described herein, reference to “at least two,” “at least five,” etc. of the biomarkers listed in any particular biomarker set means any and all combinations of the biomarkers identified. Specific biomarkers for the biomarker profile do not have to be in rank order in Table 1 but may also include any biomarker, fragment or molecular form, including those described in FIG. 7, as discussed herein.

As used herein, the term “known ovarian cancer biomarker” refers to any biomarker for which an increased or decreased expression level, as compared to a control, has previously been shown to be associated with ovarian cancer. Known ovarian cancer biomarkers include, but are not limited to, CA125, IGFBP2, WFDC2, LRG1, CLIC1, PRDX6, CTSD (including CTSD-30 kDa and CTSD-52 kDa), CLIC4, and TPM proteins including TPM1, TPM2, TPM3, TPM4.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

One skilled in the art may readily reproduce the compositions and methods described herein by use of the amino acid sequences of the biomarkers and other molecular forms, which are publicly available from conventional sources.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### II. BIOMARKERS AND BIOMARKER SIGNATURES USEFUL IN THE METHODS AND COMPOSITIONS

The “targets” of the compositions and methods of these inventions include, in one aspect, biomarkers listed in Table 1, optionally with other biomarkers identified herein, fragments, particularly unique fragments thereof, and molecular forms thereof. In certain embodiments, superior diagnostic tests for diagnosing the existence of ovarian cancer utilize at least one of the ligands that bind or complex with one of biomarkers of Table 1, or one of the fragments or molecular forms thereof. In other embodiments, superior diagnostic tests for distinguishing ovarian cancer from one of the conditions recited above utilize multiple ligands, each individually detecting a different specific target biomarker identified herein, or isoform, modified form or peptide thereof. In still other methods, no ligand is necessary, e.g., MRM assays.

In one embodiment the target biomarker of the methods and compositions described herein is agrin and agrin fragments (AGRN). The amino acid sequences for AGRN and its molecular forms are publically available, such as in GENBANK. Certain fragments of AGRN, including those described in FIG. 7, may also be useful as targets in the methods and compositions described herein. It should be understood that, depending upon the context, any reference to AGRN herein also refers to any of its peptides or molecular forms.

In one embodiment the target biomarker of the methods and compositions described herein is proteasome activator complex subunit 2 (PSME2). The amino acid sequence for PSME2 is publically available, such as in GENBANK. Certain fragments of PSME2, including those described in FIG. 7, may also be useful as targets in the methods and compositions described herein. It should be understood that, depending upon the context, any reference to PSME2 herein also refers to any of its peptides or molecular forms.

In one embodiment the target biomarker of the methods and compositions described herein is triosephosphate isomerase (TPI1). The amino acid sequence for TPI1 is publically available, such as in GENBANK. Certain fragments of TPI1, including those described in FIG. 7, may also be useful as targets in the methods and compositions described herein. It should be understood that, depending upon the context, any reference to TPI1 herein also refers to any of its peptides or molecular forms.

In one embodiment the target biomarker of the methods and compositions described herein is N(G),N(G)-dimethylarginine dimethylaminohydrolase 2 (DDAH2). The amino acid sequence for DDAH2 is publically available, such as in GENBANK. Certain fragments of DDAH2, including those described in FIG. 7, may also be useful as targets in the methods and compositions described herein. It should be understood that, depending upon the context, any reference to DDAH2 herein also refers to any of its peptides or molecular forms.

In one embodiment the target biomarker of the methods and compositions described herein is GM2 ganglioside activator protein (GM2A). The amino acid sequence for GM2A is publically available, such as in GENBANK. Certain fragments of GM2A, including those described in FIG. 7, may also be useful as targets in the methods and compositions described herein. It should be understood that, depending upon the context, any reference to GM2A herein also refers to any of its peptides or molecular forms.

In one embodiment the target biomarker of the methods and compositions described herein is 14-3-3 protein beta/alpha (YWHAB). The amino acid sequence for YWHAB is publically available, such as in GENBANK. Certain fragments of YWHAB, including those described in FIG. 7, may also be useful as targets in the methods and compositions described herein. It should be understood that, depending upon the context, any reference to YWHAB herein also refers to any of its peptides or molecular forms.

In one embodiment the target biomarker of the methods and compositions described herein is 14-3-3 protein eta (YWHAH). The amino acid sequence for YWHAH is publically available, such as in GENBANK. Certain fragments of YWHAH, including those described in FIG. 7, may also be useful as targets in the methods and compositions described herein. It should be understood that, depending upon the context, any reference to YWHAH herein also refers to any of its peptides or molecular forms.

In one embodiment the target biomarker of the methods and compositions described herein is proteasome subunit alpha type-1 (PSMA1). The amino acid sequence for PSMA1 is publically available, such as in GENBANK. Certain fragments of PSMA1, including those described in FIG. 7, may also be useful as targets in the methods and compositions described herein. It should be understood that, depending upon the context, any reference to PSMA1 herein also refers to any of its peptides or molecular forms.

In one embodiment the target biomarker of the methods and compositions described herein is proteasome subunit beta type-1 (PSMB1). The amino acid sequence for PSMB1 is publically available, such as in GENBANK. Certain fragments of PSMB1, including those described in FIG. 7, may also be useful as targets in the methods and compositions described herein. It should be understood that, depending upon the context, any reference to PSMB1 herein also refers to any of its peptides or molecular forms.

In one embodiment the target biomarker of the methods and compositions described herein is proteasome subunit beta type-2 (PSMB2). The amino acid sequence for PSMB2 is publically available, such as in GENBANK. Certain fragments of PSMB2, including those described in FIG. 7, may also be useful as targets in the methods and compositions described herein. It should be understood that, depending upon the context, any reference to PSMB2 herein also refers to any of its peptides or molecular forms.

In one embodiment the target biomarker of the methods and compositions described herein is proteasome subunit beta type-4 (PSMB4). The amino acid sequence for PSMB4 is publically available, such as in GENBANK. Certain fragments of PSMB4, including those described in FIG. 7, may also be useful as targets in the methods and compositions described herein. It should be understood that, depending upon the context, any reference to PSMB4 herein also refers to any of its peptides or molecular forms.

In one embodiment the target biomarker of the methods and compositions described herein is ferritin. The amino acid sequence for ferritin is publically available, such as in GENBANK. Certain fragments of ferritin, including ferritin heavy chain (FTH1) and ferritin light chain (FTL), and those described in FIG. 7, are also useful as targets in the methods and compositions described herein. It should be understood that, depending upon the context, any reference to ferritin, FTH1 or FTL herein also refers to any of its peptides or molecular forms.

In one embodiment the target biomarker of the methods and compositions described herein is metalloproteinase inhibitor 2 (TMP2). The amino acid sequence for TMP2 is publically available, such as in GENBANK. Certain fragments of TMP2, including those described in FIG. 7, may also be useful as targets in the methods and compositions described herein. It should be understood that, depending upon the context, any reference to TMP2 herein also refers to any of its peptides or molecular forms.

In one embodiment the target biomarker of the methods and compositions described herein is carbonic anhydrase 13 (CA13). The amino acid sequence for CA13 is publically available, such as in GENBANK. Certain fragments of CA13, including those described in FIG. 7, may also be useful as targets in the methods and compositions described herein. It should be understood that, depending upon the context, any reference to CA13 herein also refers to any of its peptides or molecular forms.

In one embodiment the target biomarker of the methods and compositions described herein is proteasome subunit beta type-10 (PSMB10). The amino acid sequence for PSMB10 is publically available, such as in GENBANK. Certain fragments of PSMB10, including those described in FIG. 7, may also be useful as targets in the methods and compositions described herein. It should be understood that, depending upon the context, any reference to PSMB10 herein also refers to any of its peptides or molecular forms.

In one embodiment the target biomarker of the methods and compositions described herein is fibroleukin (FGL2). The amino acid sequence for FGL2 is publically available, such as in GENBANK. Certain fragments of FGL2, including those described in FIG. 7, may also be useful as targets in the methods and compositions described herein. It should be understood that, depending upon the context, any reference to FGL2 herein also refers to any of its peptides or molecular forms.

In one embodiment the target biomarker of the methods and compositions described herein is peptidoglycan recognition protein 1 (PGLYRP1). The amino acid sequence for PGLYRP1 is publically available, such as in GENBANK. Certain fragments of PGLYRP1, including those described in FIG. 7, may also be useful as targets in the methods and compositions described herein. It should be understood that, depending upon the context, any reference to PGLYRP1 herein also refers to any of its peptides or molecular forms.

In still another embodiment, the biomarkers targeted by the methods and compositions described herein includes various combinations of these target biomarkers and/or fragments thereof.

In another embodiment, a combination of ligands is used to identify biomarkers including any of the known ovarian cancer biomarkers, CA125, CLIC1, PRDX6, CTSD, CLIC4, IGFPB2, WFDC2 or LRG1, or any of their molecular forms, in combination with one or more of the above-noted biomarkers of Table 1. In one embodiment, the methods and compositions employ ligands that target multiple biomarkers. The multiple biomarker combinations include, without limitation, or consist of, the following exemplary combinations of biomarkers or combinations that include different molecular forms of the same biomarker, for diagnosis of ovarian cancer or for monitoring the progression of the severity of disease or remission of disease:

One or more of PSMB4, AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1; or

One or more of PSMB4, PSMB1, PSMB2, PSMB10, AGRN, FTH1, FTL, CA13, or PGLYRP1; or

Two, 3, 4, 5, 6, 8 or all 9 of PSMB4, PSMB1, PSMB2, PSMB10, AGRN, FTH1, FTL, CA13, or PGLYRP1 with one or more of CA125, HE4, CLIC1, and CTSD or another known biomarker;

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1; or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and CA125; or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and HE4; or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and CLIC1; or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and PRDX6; or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and CTSD (CTSD-30 kDa or CTSD-52 kDa); or

PSMB4 with one or more of AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1, and an additional known ovarian cancer biomarker; or

PSMB4 with one or more of AGRN, PSME2, TPI1, DDAH2, GM2A, YWHAB, YWHAH, PSMA1, PSMB1, and PSMB2; or

PSMB4 with one or more of AGRN, PSME2, TPI1, DDAH2, GM2A, YWHAB, YWHAH, PSMA1, PSMB1, and PSMB2, and one or more of CA125, HE4, CLIC1, and CTSD; or

PSMB4 with one or more of AGRN, PSME2, TPI1, DDAH2, GM2A, YWHAB, YWHAH, PSMA1, PSMB1, and PSMB2, and an additional known ovarian cancer biomarker; or

FTH1 with one or more of FTL and TIMP2; or

FTH1 with one or more of FTL and TIMP2, and one or more of CA125, HE4, CLIC1, and CTSD; or

FTH1 with one or more of FTL and TIMP2, and an additional known ovarian cancer biomarker; or

CA13 with one or more of PSMB10, FGL2 and PGLYRP1; or

CA13 with one or more of PSMB10, FGL2 and PGLYRP1, and one or more of CA125, HE4, CLIC1, and CTSD; or

CA13 with one or more of PSMB10, FGL2 and PGLYRP1, and an additional known ovarian cancer biomarker; or

One or more of AGRN, PSME2, TPI1, and YWHAH; or

One or more of AGRN, PSME2, TPI1, YWHAH, and an additional known ovarian cancer biomarker; or

One or more of PSMA1, PSMB2, PSMB4, and PSMB10; or

One or more of PSMA1, PSMB2, PSMB4, PSMB10, and an additional known ovarian cancer biomarker.

Still other combinations of the Table 1 biomarkers can be targeted in combination with other known ovarian cancer biomarkers to produce a desired signature for one of the ovarian cancer-related conditions described above.

For example, among desirable biomarker signatures are signatures that comprise, or consist of, at least 2, 3, 4, 5, 6, 7, 8, 9, 10, 11, 12, 13, 14, 15, 16, 17 or all 18 of the biomarkers of Table 1, including optionally CA125, HE4, CLIC1, PRDX6, CTSD (CTSD-30 kDa or CTSD-52 kDa) CLIC4, IGFPB2 or LRG1 or any other known ovarian cancer biomarker or molecular forms or peptides thereof.

As further stated above, the biomarkers/biomarker signatures described above, can in another embodiment, refer to nucleic acid sequences, genes and transcripts encoding the biomarkers and expression profiles thereof.

### III. DIAGNOSTIC REAGENTS, DEVICES AND KITS

### A. Labeled or Immobilized Biomarkers or Peptides or Molecular Forms

In one embodiment, diagnostic reagents or devices for use in the methods of diagnosing ovarian cancer include one or more target biomarker or peptide fragment identified in Table 1 or FIG. 7 herein, or molecular forms thereof, associated with a detectable label or portion of a detectable label system. In another embodiment, a diagnostic reagent includes one or more target biomarker or peptide fragment thereof identified in Table 1, immobilized on a substrate. In still another embodiment, combinations of such labeled or immobilized biomarkers are suitable reagents and components of a diagnostic kit or device.

In another aspect, suitable embodiments of such labeled or immobilized reagents include at least one, 2, 3, 4, 5, 6, 7, 8, 9, 10, 11, 12, 13, 14, 15, 16, 17 or all 18 of biomarkers of Table 1 or their unique peptide fragments therein.

In one aspect the reagent, device or kit comprises or consists of ligands that individually specifically complex with, bind to, or quantitatively detect or identify multiple isoforms of any of biomarkers PSMB4, AGRN, PSME2, TPI1, DDAH2, GM2A, YWHAB, YWHAH, PSMA1, PSMB1, PSMB2, FTH1, FTL, TIMP2, CA13, PSMB10, FGL2, and PGLYRP1.

In another aspect, the reagent, device or kit comprises or consists of ligands that individually specifically complex with, bind to, or quantitatively detect or identify two or more biomarkers selected from any of the specific combinations of biomarkers recited above.

In another embodiment suitable embodiments include at least one biomarker, CA-125, or an isoform, pro-form, modified molecular form, or peptide fragment thereof.

In another embodiment suitable embodiments include at least one biomarker, CA125, CLIC1, PRDX6, CTSD, CLIC4, IGFPB2, WFDC2 or LRG1 or an isoform, pro-form, modified molecular form, or peptide fragment thereof.

Any combination of labeled or immobilized biomarkers can be assembled in a diagnostic kit or device for the purposes of diagnosing ovarian cancer, such as those combinations of biomarkers discussed herein.

For these reagents, the labels may be selected from among many known diagnostic labels, including those described above. Similarly, the substrates for immobilization in a device may be any of the common substrates, glass, plastic, a microarray, a microfluidics card, a chip, a bead or a chamber.

### B. Labeled or Immobilized Ligands that Bind or Complex with the Biomarkers

In another embodiment, the diagnostic reagent or device includes a ligand that binds to or complexes with a biomarker of Table 1 or a unique peptide thereof including those described in FIG. 7, or a molecular form thereof or a combination of such ligands.

In one embodiment, such a ligand desirably binds to a protein biomarker or a unique peptide contained therein, and can be an antibody which specifically binds a single biomarker of Table 1, or a unique peptide in that single biomarker including those described in FIG. 7. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand itself may be labeled or immobilized.

In another embodiment, suitable labeled or immobilized reagents include at least 2, 3, 4, 5, 6, 7 8, 9, 10, 11, 12, 13, 14, 15, 16, 17 or 18 or more ligands. Each ligand binds to or complexes with a single biomarker protein/peptide, fragment, or molecular form of the biomarker(s) of Table 1. In some of the embodiments in which the combination of biomarkers includes CA125, HE4, CLIC1, PRDX6, CTSD, CLIC4, IGFPB2 or LRG1 or another known additional biomarker that may be in higher abundance in serum, ligands to each of these additional biomarkers may be employed in the diagnostic reagent.

Any combination of labeled or immobilized biomarker ligands can be assembled in a diagnostic kit or device for the purposes of diagnosing ovarian cancer.

Thus, a kit or device can contain multiple reagents or one or more individual reagents. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items.

The diagnostic reagents, devices, or kits compositions based on the biomarkers of Tables 1 or fragments thereof described herein, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, Mass spectrometry quantitative assays, or PCR, RT-PCR or Q PCR techniques.

In one embodiment, a kit includes multiple antibodies directed to bind to one or more of the combinations of biomarkers described above, wherein the antibodies are associated with detectable labels.

In another embodiment, the reagent ligands are nucleotide sequences, the diagnostic reagent is a polynucleotide or oligonucleotide sequence that hybridizes to gene, gene fragment, gene transcript or nucleotide sequence encoding a biomarker of Table 1 or encoding a unique peptide thereof including those described in FIG. 7. Such a polynucleotide/oligonucleotide can be a probe or primer, and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker of Table 1, optionally including one or more additional known biomarkers, such as CA125, HE4, CLIC1, PRDX6, CTSD, CLIC4, IGFPB2 or LRG1. For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 50 and 80° C., e.g. about 50 to 70° C. are typically preferred.

The selection of the ligands, biomarker sequences, their length, suitable labels and substrates used in the reagents and kits are routine determinations made by one of skill in the art in view of the teachings herein of which biomarkers form signature suitable for the diagnosis of ovarian cancer.

### IV. METHODS FOR DIAGNOSING OR MONITORING OVARIAN CANCER

In another embodiment, a method for diagnosing or detecting or monitoring the progress of ovarian cancer in a subject comprises, or consists of, a variety of steps.

### A. Sample Preparation

The test sample is obtained from a human subject who is to undergo the testing or treatment. The subject's sample can in one embodiment be provided before initial diagnosis, so that the method is performed to diagnose the existence of an ovarian cancer. In another embodiment, depending upon the reference standard and markers used, the method is performed to diagnose the stage of ovarian cancer. In another embodiment, depending upon the reference standard and markers used, the method is performed to diagnose the type or subtype of ovarian cancer from the types and subtypes identified above. In another embodiment, the subject's sample can be provided after a diagnosis, so that the method is performed to monitor progression of an ovarian cancer. In another embodiment, the sample can be provided prior to surgical removal of an ovarian tumor or prior to therapeutic treatment of a diagnosed ovarian cancer and the method used to thereafter monitor the effect of the treatment or surgery, and to check for relapse. In another embodiment, the sample can be provided following surgical removal of an ovarian tumor or following therapeutic treatment of a diagnosed ovarian cancer, and the method performed to ascertain efficacy of treatment or relapse. In yet another embodiment the sample may be obtained from the subject periodically during therapeutic treatment for an ovarian cancer, and the method employed to track efficacy of therapy or relapse. In yet another embodiment the sample may be obtained from the subject periodically during therapeutic treatment to enable the physician to change therapies or adjust dosages. In one or more of these embodiments, the subject's own prior sample can be employed in the method as the reference standard.

Preferably where the sample is a fluid, e.g., blood, serum or plasma, obtaining the sample involves simply withdrawing and preparing the sample in the traditional fashion for contact with the diagnostic reagent. Where the sample is a tissue or tumor sample, it may be prepared in the conventional manner for contact with the diagnostic reagent. Where the sample is a tumor secretome, the sample may be prepared as described in the examples below.

The method further involves contacting the sample obtained from a test subject with a diagnostic reagent as described above under conditions that permit the reagent to bind to or complex with one or more biomarker(s) of Table 1 which may be present in the sample. This method may employ any of the suitable diagnostic reagents or kits or compositions described above.

### B. Measuring Biomarker Levels

Thereafter, a suitable assay is employed to detect or measure in the sample the protein level (actual or relative) of one or more biomarker(s) of Table 1. Alternatively, a suitable assay is employed to generate a protein abundance profile (actual or relative or ratios thereof) of multiple biomarkers of Table 1 from the sample or of multiple different molecular forms of the same biomarker or both. In another embodiment, the above method further includes measuring in the biological sample of the subject the protein level of an additional biomarker, such as CA125, CLIC1, PRDX6, CTSD, CLIC4, IGFPB2, WFDC2 (HE4) or LRG1 or other known ovarian cancer biomarker. In another embodiment, the above method further includes measuring in the biological sample of the subject the protein levels of two or more additional biomarkers which form a biomarker protein abundance signature for ovarian cancer.

The measurement of the biomarker(s) in the biological sample may employ any suitable ligand, e.g., antibody, antibody mimic or equivalent (or antibody to any second biomarker) to detect the biomarker protein. Such antibodies may be presently extant in the art or presently used commercially, such as those available as part of commercial antibody sandwich ELISA assay kits or that may be developed by techniques now common in the field of immunology. A recombinant molecule bearing the binding portion of a biomarker antibody, e.g., carrying one or more variable chain CDR sequences that bind e.g., PSMB4, AGRN, PSME2, TPI1, DDAH2, GM2A, YWHAB, YWHAH, PSMA1, PSMB1, PSMB2, FTH1, FTL, TIMP2, CA13, PSMB10, FGL2, PGLYRP1, etc. may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers of Table 1 or a particular sequence of the selected biomarker as defined in Table 1 may be employed in the methods described herein, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and devices of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochrome, e.g., fluorescein isothiocyanate (FITC), phycoerythrin (PE), allophycocyanin (APC), coriphosphine-O(CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays and devices of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker antibodies with optional labels for use in this invention is within the skill of the art, provided with this specification, the documents incorporated herein, and the conventional teachings of immunology.

Similarly the particular assay format used to measure the selected biomarker in a biological sample may be selected from among a wide range of protein assays, such as described in the examples below. Suitable assays include enzyme-linked immunoassays, sandwich immunoassays, homogeneous assays, immunohistochemistry formats, or other conventional assay formats. In one embodiment, a serum/plasma sandwich ELISA is employed in the method. In another embodiment, a mass spectrometry-based assay is employed. In another embodiment, a MRM assay is employed, in which antibodies are used to enrich the biomarker in a manner analogous to the capture antibody in sandwich ELISAs.

One of skill in the art may readily select from any number of conventional immunoassay formats to perform this invention.

Other reagents for the detection of protein in biological samples, such as peptide mimetics, synthetic chemical compounds capable of detecting the selected biomarker may be used in other assay formats for the quantitative detection of biomarker protein in biological samples, such as high pressure liquid chromatography (HPLC), immunohistochemistry, etc.

Employing ligand binding to the biomarker proteins or multiple biomarkers forming the signature enables more precise quantitative assays, as illustrated by the multiple reaction monitoring (MRM) mass spectrometry (MS) assays. As an alternative to specific peptide-based MRM-MS assays that can distinguish specific protein isoforms and proteolytic fragments, the knowledge of specific molecular forms of biomarkers allows more accurate antibody-based assays, such as sandwich ELISA assays or their equivalent. Frequently, the isoform specificity and the protein domain specificity of immune reagents used in pre-clinical (and some clinical) diagnostic tests are not well defined. MRM-MS assays were used to quantitative the levels of a number of the low abundance biomarkers in samples, as discussed in the examples.

In one embodiment, suitable assays for use in these methods include immunoassays using antibodies or ligands to the above-identified biomarkers and biomarker signatures. In another embodiment, a suitable assay includes a multiplexed MRM based assay for two more biomarkers that include one or more of the proteins/unique peptides in Table 1. It is anticipated that ultimately the platform most likely to be used in clinical assays will be multi-plexed or parallel sandwich ELISA assays or their equivalent, primarily because this platform is the technology most commonly used to quantify blood proteins in clinical laboratories. MRM MS assays may continue to be used productively to help evaluate the isoform/molecular form specificity of any existing immunoassays or those developed in the future.

### C. Detection of a Change in Biomarker Abundance Level and Diagnosis

The protein level of the one or more biomarker(s) in the subject's sample or the protein abundance profile of multiple said biomarkers as detected by the use of the assays described above is then compared with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the protein level of a biomarker or biomarkers in the subject's sample from the same biomarker or biomarkers in the reference standard can indicate an appropriate diagnosis. An appropriate diagnosis can be identifying a risk of developing ovarian cancer, a diagnosis of ovarian cancer (or stage or type thereof), a diagnosis or detection of the status of progression or remission of ovarian cancer in the subject following therapy or surgery, a determination of the need for a change in therapy or dosage of therapeutic agent. The method is thus useful for early diagnosis of disease, for monitoring response or relapse after initial diagnosis and treatment or to predict clinical outcome or determine the best clinical treatment for the subject.

In one embodiment, the change in protein level of each biomarker can involve an increase of a biomarker or multiple biomarkers in comparison to the specific reference standard. In one embodiment, a selection or all of the biomarkers of Table 1 are increased in a subject sample from a patient having ovarian cancer when compared to the levels of these biomarkers from a healthy reference standard. In another embodiment, a selection or all of the biomarkers of Table 1 are increased in a subject sample from a patient having ovarian cancer prior to therapy or surgery, when compared to the levels of these biomarkers from a post-surgery or post-therapy reference standard.

In another embodiment, the change in protein level of each biomarker can involve a decrease of a biomarker or multiple biomarkers in comparison to the specific reference standard. In one embodiment, a selection or all of the biomarkers of Table 1 are decreased in a subject sample from a patient having ovarian cancer following surgical removal of a tumor or following chemotherapy/radiation when compared to the levels of these biomarkers from a pre-surgery/pre-therapy ovarian cancer reference standard or a reference standard which is a sample obtained from the same subject pre-surgery or pre-therapy.

In still other embodiments, the changes in protein levels of the biomarkers may be altered in characteristic ways if the reference standard is a particular type of ovarian cancer, e.g., serous, epithelial, mucinous or clear cell, or if the reference standard is derived from benign ovarian cysts or nodules.

The results of the methods and use of the compositions described herein may be used in conjunction with clinical risk factors to help physicians make more accurate decisions about how to manage patients with ovarian cancers. Another advantage of these methods and compositions is that diagnosis may occur earlier than with more invasive diagnostic measures.

### D. Alternative Embodiments

In an alternative embodiment, the method of diagnosis or risk of diagnosis involves using the nucleic acid hybridizing reagent ligands described above to detect a significant change in expression level of the subject's sample biomarker or biomarkers from that in a reference standard or reference expression profile which indicates a diagnosis, risk, or the status of progression or remission of ovarian cancer in the subject. These methods may be performed in other biological samples, e.g., biopsy tissue samples, tissue removed by surgery, or tumor cell samples, including circulating tumor cells isolated from the blood, to detect or analyze a risk of developing an ovarian cancer, as well as a diagnosis of same. Such methods are also known in the art and include contacting a sample obtained from a test subject with a diagnostic reagent comprising a ligand which is a nucleotide sequence capable of hybridizing to a nucleic acid sequence encoding a biomarker of Table 1, said ligand associated with a detectable label or with a substrate. Thereafter one would detect or measure in the sample or from an expression profile generated from the sample, the expression levels of one or more of the biomarkers or ratios thereof. The expression level(s) of the biomarker(s) in the subject's sample or from an expression profile or ratio of multiple said biomarkers are then compared with the expression level of the same biomarker or biomarkers in a reference standard. A significant change in expression level of the subject's sample biomarker or biomarkers from that in the reference standard indicates a diagnosis, risk, or the status of progression or remission of ovarian cancer in the subject.

Suitable assay methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The comparison of the quantitative or relative expression levels of the biomarkers may be done analogously to that described above for the comparison of protein levels of biomarkers.

### E. Non-Ligand-Based Analysis

In another aspect, a method for diagnosing or detecting or monitoring the progress of ovarian cancer in a subject involves non-ligand based methods, such as mass spectrometry. For example, proteins in a biological sample obtained from a test subject may be contacted with a chemical or enzymatic agent and the proteins, including the biomarkers contained therein fragmented in the sample. The digested sample or portions thereof are injected into a mass spectrometer and the protein levels or ratios of one or more of the biomarkers of Table 1, optionally with other known biomarkers, modified molecular forms, peptides and unique peptides or ratios thereof, are quantitatively identified or measured by mass spectrometry. The protein levels of the biomarkers in the subject's sample are then compared with the level of the same biomarker or biomarkers in a reference standard or to a predetermined cutoff derived from the reference standard. In one embodiment, the agent is a proteolytic enzyme. In another embodiment, the agent is trypsin.

A significant change in protein level of the subject's sample biomarker or biomarkers from that in the reference standard or from a predetermined cutoff indicates a diagnosis, risk, or the status of progression or remission of ovarian cancer in the subject.

Thus, the various methods, devices and steps described above can be utilized in an initial diagnosis of ovarian cancer or other ovarian condition, as well as in clinical management of patients with ovarian cancer after initial diagnosis. Uses in clinical management of the various devices, reagents and assay methods, include without limitation, monitoring for reoccurrence of disease or monitoring remission or progression of the cancer and either before, during or after therapeutic or surgical intervention, selecting among therapeutic protocols for individual patients, monitoring for development of toxicity or other complications of therapy, and predicting development of therapeutic resistance.

In one embodiment, the method involves enriching the biomarker protein or one or more peptides produced by specific proteolysis in the sample by contacting the sample with an antibody prior to injecting into a mass spectrometer in a manner analogous to a capture antibody in a conventional sandwich ELISA. In another embodiment, the method involves depleting the sample of non-target proteins prior to injecting sample into a mass spectrometer. The depletion may also be performed using antibodies to the non-targets. The method described herein may use liquid chromatographic mass spectrometry, such as HPLC. One such method is described in detail in the Examples below.

### V. EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1

### Materials and Methods

Reagents. Molecular-biology-grade ethanol (200 proof); LC-MS-grade formic acid; sodium phosphate monobasic; N,N-dimethylacrylamide (DMA), ammonium bicarbonate; and iodoacetamide were purchased from Sigma-Aldrich (St. Louis, Mo.). Sodium dodecyl sulfate (SDS), 2-mercaptoethanol, and Tris were purchased from Bio-Rad (Hercules, Calif.). ZOOM focusing buffers and thiourea were obtained from Invitrogen (Carlsbad, Calif.). PlusOne reagents dithiothreitol (DTT), 3-[(3-cholamidopropyl)dimethylammonio]-1-propanesulfonate (CHAPS), and urea were purchased from GE Healthcare (Piscataway, N.J.). HPLC-grade acetonitrile was purchased from Thomas Scientific (Swedesboro, N.J.). Tris(2-carboxyethyl)phosphine (TCEP) was obtained from Pierce (Rockford, Ill.), and sequencing-grade modified trypsin was purchased from Promega (Madison, Wis.).

Cell culture. The human EOC cell line OVCAR-3 was obtained from the American Type Culture Collection (ATCC, Manassas, Va.). The cells were maintained in a 37° C. incubator with a 5% CO₂-95% air atmosphere in RPMI-1640 medium (ATCC) supplemented with 0.01 mg/ml bovine insulin and 10% fetal calf serum.

Ovarian cancer growth in vivo. Nine severe combined immunodeficiency (SCID) mice were injected subcutaneously into the flank with 50 μA of OVCAR-3 cells (2×10⁶) mixed 1:1 with 50 μl Matrigel (BD Biosciences, San Jose, Calif.). Tumors were allowed to grow until final tumor size was estimated to be at least 1 cm³ using calipers, but less than 10% of body weight. Blood was collected, mice were euthanized, and tumors were removed at 12 weeks post-injection. Necrosis of tumor tissue was assessed by microscopic inspection of hematoxylin and eosin- (H&E) stained, paraffin-embedded sections (5 μm). The study protocol was approved by The Wistar Institute's Institutional Animal Care and Use Committee (IACUC).

SCID mouse plasma. Blood was collected from SCID mice by cardiac puncture under anesthesia into Microtainer 0.5 ml K₂ EDTA Blood Collection Tubes (Becton Dickinson, Franklin Lakes, N.J.) and centrifuged for 3 min at RT. Individual aliquots of plasma from each mouse were snap-frozen and stored at −80° C. Plasma from selected mice were subsequently thawed and pooled based on an assessment of tumor size and minimal necrosis. The pooled plasma was re-aliquoted, snap-frozen, and stored at −80° C. until future use. Total protein concentration of pooled plasma was measured using a BCA Protein Assay (Pierce).

Tumor secretome isolation. Immediately after removing tumors from the mouse, a section of fresh ovarian tumor tissue was cut into small pieces (2-3 mm³), placed in the upper chamber of a 5 μm PVDF microcentrifuge filter (Millipore, Billerica, Mass.), and washed three times with 400 μL of PBS for 1 min each. The tissue sections were then transferred to the upper chamber of a 0.22 μm PVDF microcentrifuge filter and incubated in 400 μL of serum-free RPMI-1640 medium for 2 h in 5% CO₂, 95% air at 37° C. After incubation, the supernatant (conditioned media) was recovered by centrifugation, then frozen and stored at −80° C. until needed. For secretome analysis, 500 μL aliquots of conditioned media from tumors from eight mice were thawed, pooled in batches of four mice each, and concentrated to ˜30 μL by ultrafiltration using a 10K MWCO concentration unit. Membrane rinses using 1% SDS, 50 mM Tris, pH 8.5 were combined with the concentrated sample to maximize protein recovery.

Human serum. Human sera from patients with benign ovarian tumors, early-stage ovarian cancer, and from late-stage ovarian cancer patients were collected before clinical treatment at approximately the time of diagnosis at the University of Turin, Turin, Italy, and control serum samples were collected from healthy, postmenopausal females at The Wistar Institute, as previously described.¹⁸ All patient specimens were collected with full patient consent and in accordance with Institutional Review Board (IRB) and Health Insurance Portability and Accountability Act (HIPAA) requirements.

Immunoaffinity removal of major blood proteins. Mouse plasma was depleted using a 4.6×100 mm MARS Mouse-3 HPLC column (Agilent Technologies, Wilmington, Del.), essentially as previously described,¹⁸ with the exception that a total of 400 μL of pooled plasma was diluted five-fold with equilibration buffer, filtered through a 0.22 m microcentrifuge filter, and applied to the antibody column in eight serial injections of 250 μl per depletion. Human serum samples (typically 30-60 μL) were depleted of the 20-most-abundant serum proteins using a ProteoPrep20 Immunodepletion Column (Sigma), as described previously.¹⁹

MicroSol-IEF fractionation. Immunodepleted and concentrated mouse plasma (2.2 mg) was fractionated by microscale solution IEF (MicroSol-IEF) as previously described,^{18, 20-21} using a ZOOM-IEF fractionator (Invitrogen) where the separation chambers were defined by immobilized gel membranes having pH values of 3.0, 4.6, 5.4, 6.2, and 12.0, respectively.

SDS-PAGE/in-gel trypsin digestion. After evaluation of the MicroSol-IEF separation on analytical SDS gels, concentrated samples were loaded onto multiple lanes of pre-cast 12% Bis-Tris NuPAGE gels (Invitrogen) and separated for discrete distances (1, 4, or 6 cm) by SDS-PAGE. Gels were stained with Colloidal Blue (Invitrogen), each gel lane was sliced into uniform 1 mm slices, and corresponding slices from triplicate lanes were combined in a single well of a 96-well pierced digestion plate (Bio-Machines, Inc., Carrboro, N.C.) and digested overnight with 0.02 μg/μL of modified trypsin, as previously described.^{19, 22}

LC-MS/MS. Tryptic digests were analyzed using an LTQ-Orbitrap XL mass spectrometer (Thermo Scientific, Waltham, Mass.) interfaced with a Nano-ACQUITY UPLC system (Waters, Milford, Mass.), as described previously.¹⁹ For each tryptic digest, 8 μL was injected onto a UPLC Symmetry trap column (180 μm i.d.×2 cm packed with 5 μm C18 resin; Waters), and tryptic peptides were separated by RP-HPLC on a BEH C18 nanocapillary analytical column (75 μm i.d.×25 cm, 1.7 μm particle size; Waters). The mass spectrometer was set to scan m/z from 400 to 2000. The full MS scan was collected at 60,000 resolution in the Orbitrap in profile mode followed by data-dependant MS/MS scans on the six-most-abundant ions exceeding a minimum threshold of 1000 collected in the linear trap. Monoisotopic precursor selection was enabled and charge-state screening was enabled to reject z=1 ions. Ions subjected to MS/MS were excluded from repeated analysis for 60 s.

Data processing. MS/MS spectra were extracted and searched using the SEQUEST algorithm (v. 28, rev. 13, University of Washington, Seattle, Wash.) in Bioworks (v. 3.3.1, Thermo Scientific) against a combined human and mouse UniRef100 protein sequence database (v. June 2011) to which commonly observed “contaminants” (trypsin, keratins, etc.) were added. A decoy database was produced by reversing the protein sequence of each database entry, and the entire reversed database was appended in front of the forward human and mouse databases, respectively. Spectra were searched with a partial tryptic constraint of up to two missed cleavages, 100 ppm precursor mass tolerance, 1 Da fragment ion mass tolerance, static modification of cys (+99.06840 for samples alkylated with DMA or +57.0215 Da for samples alkylated with IAM), and variable modification of methionine (+15.9949). The use of a partial tryptic constraint and 100 ppm precursor tolerance for the database search had previously been shown to enhance depth of analysis for serum and plasma proteome analysis.²³ Consensus protein lists were created using DTASelect (v. 2.0, licensed from Scripps Research Institute, La Jolla, Calif.) and the following filters were applied: full tryptic constraint, mass accuracy ≦10 ppm, and ΔCn≧0.05.²³ FDR was estimated from the ratio of unique peptides matching reverse sequences to the number of unique peptides matching forward sequences. Non-redundant peptide totals derived from DTASelect and used for FDR calculations include variable modifications and different charge states as separate peptides. Different charge states and variable modifications of methionine oxidation were collapsed into a single unique peptide count, and peptides shared among multiple proteins were assigned to the protein having the highest sequence coverage, as previously described.¹⁸

Proteins identified in the database search were sorted into “human,” “mouse,” or “indistinguishable” based upon their species-specific sequences, as previously described.¹⁸ To confirm species-specific assignments, putative uniquely human and mouse sequences were searched against the mouse and human UniRef100 databases (Jun. 20, 2011), respectively, using BLAST. Keratins and other presumed contaminants were removed from the entire dataset.

The secretome and plasma datasets were compared to identify common and unique proteins. Specifically, all protein and peptide data were placed in a relational database (MySQL) and identifications were matched based on UniRef100 protein accession numbers.

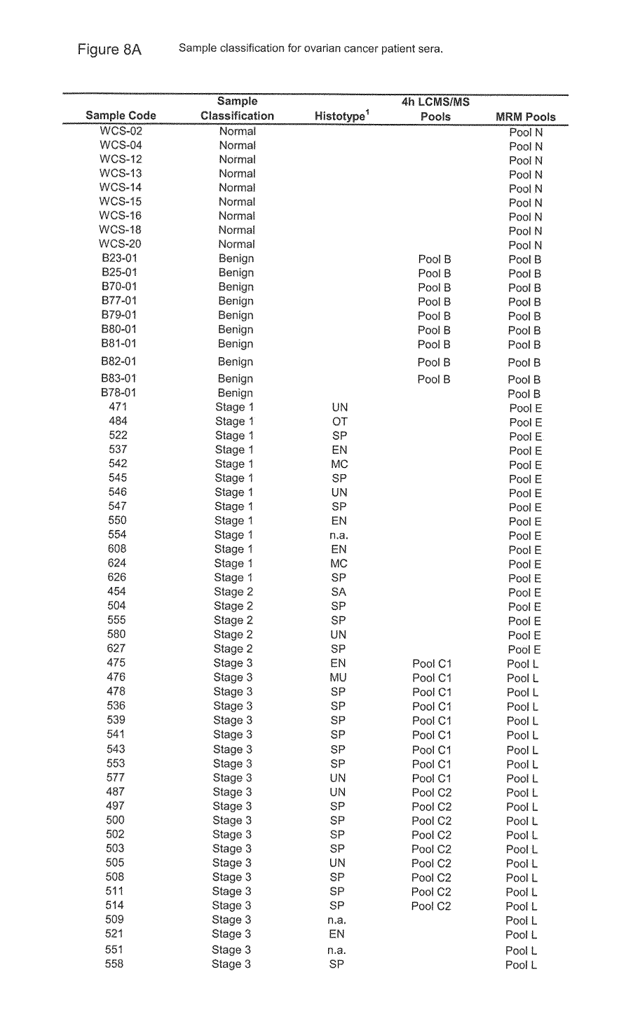

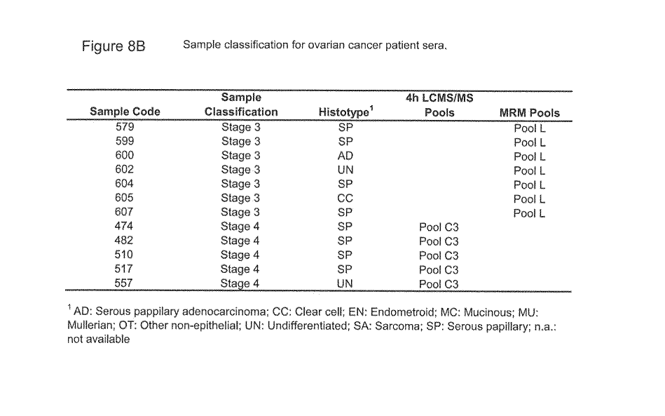

Label-free quantitation of patient serum pools. To determine whether candidate biomarkers could be detected in ovarian cancer patient sera, two levels of label-free comparisons of pooled sera were performed. One pool of serum from benign patients and three pools of advanced ovarian cancer patient serum samples were made as described in FIG. 8. Pools were immunodepleted and separated on a 1D SDS gel, each serum proteome was separated into 40 fractions, and each slice was digested with trypsin and analyzed by LC-MS/MS using a 4 h gradient at 200 mL/min consisting of 5-28% B over 168 min, 28-50% B over 51.5 min, 50-80% B over 5 min, and 80% B for 4.5 min, before returning to 5% B over 0.5 min. A short blank gradient was run in between samples to minimize carryover. Full-MS and LC-MS/MS data from fractions 17-32, which encompassed the 8-50 kDa region of the gel, were analyzed and searched against the human UniRef100 protein sequence database (v. June 2011) plus common contaminants and processed, as described above. Total spectral counts observed for each protein were compared across serum pools, as previously described.²⁴

Subsequently, a more refined quantitative analysis for selected candidate biomarkers was performed using Rosetta Elucidator software (version 3.3, Rosetta Biosoftware, Seattle, Wash.) to compare peptide signal intensities in full MS scans. Based on peptide elution profiles and ion signal density, data for this label-free comparison was trimmed to 16-200 min. Retention time (RT) alignment, feature identification (discrete ion signals), feature extraction, and protein identifications were performed by the Elucidator system as previously described.^{19, 24-25}

Label-free multiple reaction monitoring. MRM experiments were performed on a 5500 QTRAP hybrid triple quadrupole/linear ion trap mass spectrometer (AB Sciex, Foster City, Calif.) interfaced with a Nano-ACQUITY UPLC system with the column heater maintained at 45° C. Tryptic digests were injected using the partial loop injection mode onto a UPLC Symmetry trap column (180 μm i.d.×2 cm packed with 5 μm C18 resin), and then separated by RP-HPLC on a BEH C18 nanocapillary analytical column (75 mm i d×25 cm, 1.7 mm particle size; Waters). Chromatography was performed with solvent A, consisting of Milli-Q water with 0.1% formic acid, and solvent B, as acetonitrile with 0.1% formic acid. Peptides and transitions used for quantitation were selected from discovery results (typically the mouse tumor secretome) and further verified by MRM-initiated detection and sequencing (MIDAS) using the 5500 QTRAP mass spectrometer. MIDAS experiments were performed at 200 mL/min with a 77-min gradient consisting of 5-28% B over 42 min, 28-50% B over 25.5 min, 50-80% B over 5 min, and 80% B for 4.5 min, before returning to 5% B over 0.5 min. To increase throughput, after optimal peptides and transitions were established, label-free MRM assays were performed with a 41-min gradient, in which peptides were eluted at 400 mL/min for 5-35% B over 38 min and 35% B for 3 min, before returning to 5% B over 0.5 min.

MRM data were acquired with a spray voltage of 3300 V, curtain gas of 20 p.s.i., nebulizer gas of 10 p.s.i., interface heater temperature of 150° C., and a pause time of 3 ms. Multiple MRM transitions were monitored per peptide at unit resolution in both Q1 and Q3 quadrupoles to maximize specificity. Scheduled MRM was used to reduce the number of concurrent transitions and maximize the dwell time for each transition. The detection window was set at 2 min, and the target scan time was set at 1.8 s. Data analysis was performed using Skyline v.1.2.²⁵ The transition with the strongest signal for each peptide was used for quantification unless interference from the matrix was observed. In these cases, another transition free of interference was chosen for quantification.

### Example 2

### Xenograft Mouse Model

The primary experimental systems where migration into the blood is assured are mouse models. One of these approaches is to identity quantitative differences in plasma or serum of genetically engineered mice bearing murine ovarian tumors compared with appropriate controls.¹⁷ Importantly, an underutilized alternative is the xenograft mouse EOC model, where in-depth proteome analysis can unambiguously identify human proteins shed by the tumor into the murine blood based upon species differences in peptide sequences found in serum or plasma.

We recently demonstrated that an in-depth 4D analysis of serum from SCID mice bearing ovarian tumors formed by an endometrial ovarian cancer cell line could detect more than a hundred human proteins that were identified by two or more peptides per protein.¹⁸ Furthermore, pilot validation of selected candidate biomarkers demonstrated that many of these proteins could be detected in human serum using multiple reaction monitoring (MRM) analysis—and three of these biomarkers were shown to be significantly elevated in cancer patients compared with normal donors.¹⁸ The advantages of using the xenograft mouse model system like genetically engineered mice include a higher tumor-to-blood-volume ratio than patients, as well as homogeneous genetic backgrounds, environment, and diet to minimize confounding factors. As noted above, a unique feature of the xenograft mouse model is the capacity to unambiguously identify proteins shed by the tumor into blood based upon detection of unique human sequences.

The current study compares mouse plasma and corresponding xenografted tumor secretomes for detection of novel ovarian cancer biomarkers. Far more human proteins are detected in the tumor secretome, but the portion of these proteins that diffuse into the blood is unknown. Hence, the primary value of tumor secretome data is to verify less-certain identifications of human proteins in mouse plasma and identify additional proteotypic peptides for subsequent quantitative multiple reaction monitoring (MRM) assays. This integrated approach identified hundreds of biomarker candidates. To streamline verification and validation in patient blood and to increase the percentage of tested candidates that are promising biomarkers, we initially used an in-depth, label-free quantitative comparison of several ovarian cancer serum pools. Only proteins that could be detected in human serum and exhibited increases in ovarian cancer compared with benign conditions advanced to MRM assay development. Pilot MRM validation of larger serum pools and comparison to known biomarkers suggest all 18 novel biomarkers selected by this process are useful in detection of ovarian cancer.

In-depth proteome analysis of plasma from SCID mice bearing ovarian tumors formed by a serous ovarian cancer line was compared with the corresponding tumor secretome. Selected high-priority candidate biomarkers were then compared to a dataset from label-free quantitative comparison of serum pools from cancer patients with advanced-stage EOC or benign controls. Technologically, this allowed us to identify those proteins that could be detected in human serum after only moderate fractionation. Biologically, it yielded insights into those proteins elevated in advanced cancer serum compared with benign ovarian conditions. Only those proteins that showed elevated levels in ovarian cancer patients were selected for development of MRM assays. This strategy proved to be highly efficient, as all proteins selected for MRM showed substantial differences between cancer and controls, suggesting that all 18 novel biomarkers identified in this study are useful in detection of ovarian cancer.

### Example 3

### Discovery, Verification, and Validation of EOC Biomarkers Using a Xenograft EOC Mouse Model

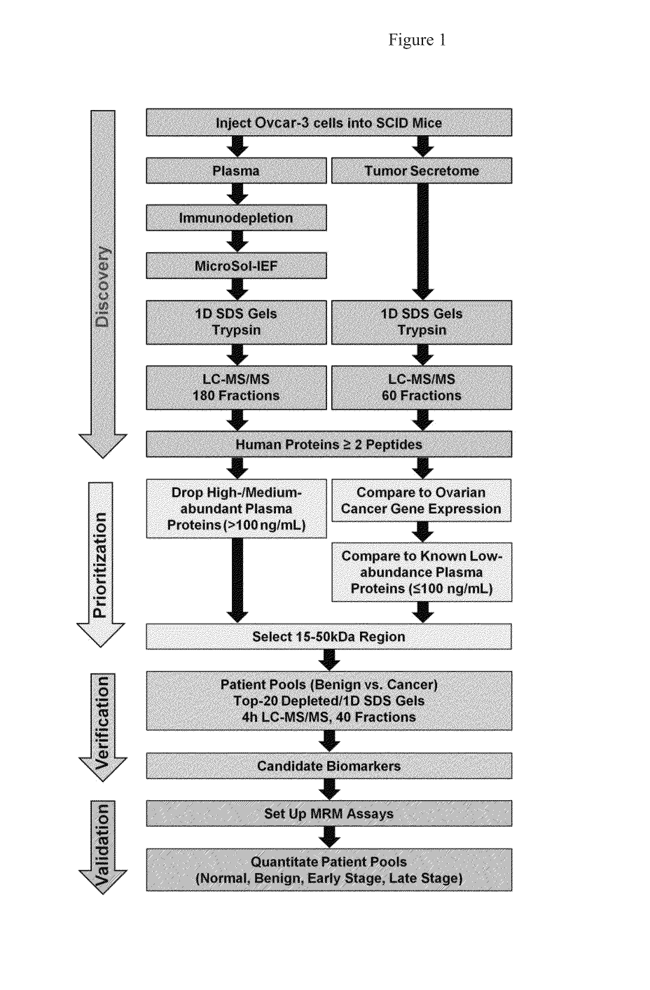

The strategies used to compare the merits of analyzing a xenograft mouse plasma proteome and/or the corresponding tumor secretome to discover novel ovarian cancer biomarkers, as well as downstream verification and initial validation, are outlined in FIG. 1. In the discovery phase, OVCAR-3, an established serous cell line, was grown in SCID mice. Tumors were excised, briefly cultured in serum-free media, and supernatants were divided into 60 fractions and analyzed using GeLC-MS/MS. Due to its greater complexity, the xenograft mouse plasma was subjected to more extensive fractionation (180 fractions) using a 4D plasma proteome separation method developed in our laboratory.²² Human proteins from the xenograft plasma and tumor secretome were prioritized, as described below, and the 15-50 kDa region of the gel was selected for proof-of-principle verification and initial validation studies because this region of the gel was observed to have the highest concentration of putative biomarkers in this study (data not shown). The verification strategy utilized a separate discovery-based, label-free quantitation of ovarian cancer patient serum pools to determine which high-priority candidate biomarkers could be detected in patient serum. These results also identified those candidate biomarkers showing elevated protein intensities in late-stage ovarian cancer serum pools. Only proteins meeting these criteria advanced to subsequent MRM validation experiments.

### Example 4

### Comparison of the Xenografted Tumor Secretome and Host Mouse Plasma Proteome

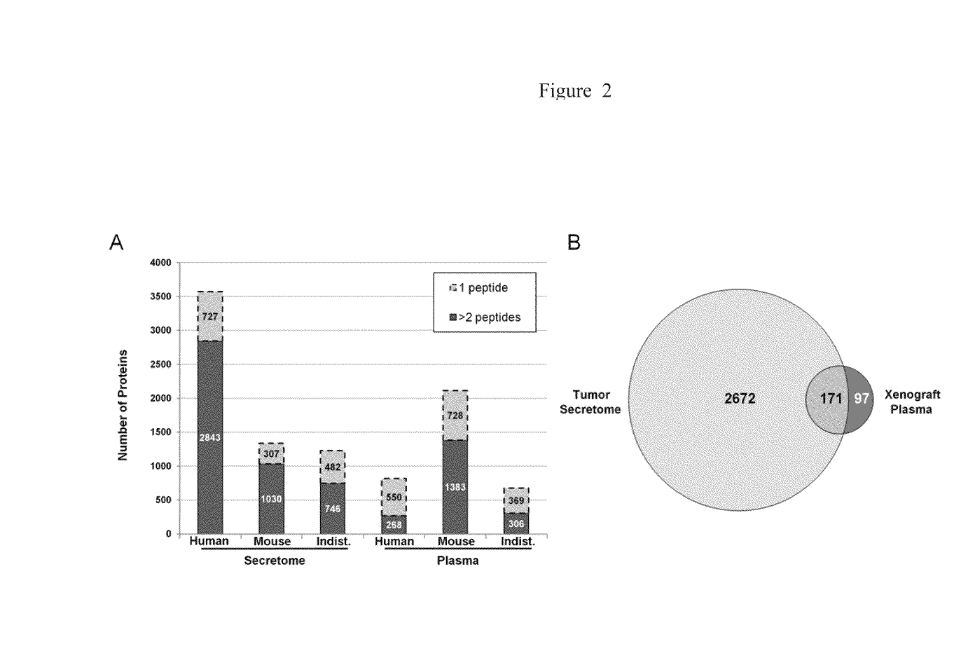

Analysis of the 60 fractions from conditioned media resulted in 487,076 MS/MS spectra, which were searched against a combined mouse and human database. A total of 6066 unique proteins were identified from 46,111 peptides at a peptide FDR of 0.9%. Eliminating single peptide proteins resulted in 4619 unique protein entries, with a peptide FDR of 0.03%. This list of high-confidence proteins (≧2 peptides) identified from the combined human and mouse dataset is listed is not shown. The complete dataset was divided into “human” and “mouse” based upon the presence of at least one peptide unique to that species, while “indistinguishable” proteins contained only peptides common to both species. A total of 2843 human proteins were identified by two or more peptides, and an additional 727 human proteins were identified by single peptides (FIG. 2A).

Analysis of the more extensively fractionated tumor-bearing mice plasma (180 fractions) resulted in acquisition of >1.1 million spectra, which were searched and processed in the same manner as the secretome dataset. A total of 3647 non-redundant human and mouse proteins were initially identified by 22,890 peptides at a peptide FDR of 5.7% for all proteins, and a 0.5% FDR for protein identifications with two or more peptides. After species classification, 268 human proteins were identified by two or more peptides and an additional 550 by a single peptide (FIG. 2A). Because the FDR was considerably lower for proteins with ≧2 peptides in both the plasma and secretome samples, we only considered proteins having two or more peptide identifications for downstream analyses.

The organ culture secretome dataset provided a much greater depth of analysis despite the less extensive fractionation used, but it is not apparent how many of these proteins are actually shed into the blood. Comparison of the overlap for the two proteomes showed that only about two-thirds of the proteins identified in the xenograft plasma samples were detected in the secretome (FIG. 2B). It was surprising that 97 proteins appeared to be uniquely identified in the mouse plasma but were not detected in the tumor secretome despite the far larger number of human proteins identified in the latter sample. These plasma proteins were further examined to identify potential reasons why they were not observed in the tumor secretome. This analysis revealed that a few proteins were additional contaminants having ambiguous protein descriptions that were missed based upon the sequence tag for contaminants, such as hypothetical proteins that are actually keratins. Others were high- to medium-abundant plasma proteins or hemoglobins that are unlikely to be specific to ovarian cancer and may have been identified as an alternative isoform in the secretome dataset. Additionally, some of these proteins were detected in the secretome dataset but all peptides were common to a mouse homolog and, hence, they were classified as “indistinguishable” in the secretome dataset. Also, a subset of proteins was discovered in the secretome as a different isoform or as a fragment of the protein identified in the mouse plasma and, therefore, had a different protein accession that was not matched in the initial dataset comparison. Finally, a few proteins were identified in the secretome by a single peptide. Ultimately, 48 of the 97 proteins apparently unique to the plasma dataset could not be matched to the secretome dataset or be explained by the above factors. Although this is only 18% of the human proteins detected in the mouse plasma, it is still surprising that such a large number of proteins were not identified in the more extensive secretome dataset. One possible reason is that some of these proteins may bind to the plastic in the culture dish, tissue debris from the mincing step, or cells that they would normally not encounter in an intact mouse. Another possibility is that some of these proteins may be shed at very low rate per hour or have a very short half-life in culture media, but accumulate with a long half-life in the plasma. That is, the tumor secretome measures proteins shed over 2 h, while some proteins may accumulate in the mouse blood with half-lives of several weeks. Finally, some of these proteins may be misclassified as human because most of them contain a single apparent human peptide and one or more common peptides, but the apparent human peptide may actually be a mouse peptide with a single nucleotide polymorphism (SNP), deamidation, or an unknown mouse sequence variant that matched the human sequence.

### Example 5

### Analysis of the Tumor Secretome Increases Sequence Coverage and Potential Candidates

Although over 2800 human proteins from the tumor were identified in the secretome, criteria for prioritizing potential plasma biomarker candidates using such a large dataset are not well defined. It seems likely that a much larger percentage of the xenograft plasma proteins will be potential blood biomarkers in patients, as those proteins do appear in the blood in this model system. However, there is more uncertainty about the species assignment for many of the apparently human proteins in the plasma compared with the secretome as discussed above. Also, some identified proteins represent a protein family rather than a unique isoform. Therefore, analysis of the secretome in parallel with the mouse plasma can be used to increase confidence of species assignment and distinguish some isoforms in the xenograft plasma samples by using the more extensive peptide coverage in the tumor secretome for most shared proteins.

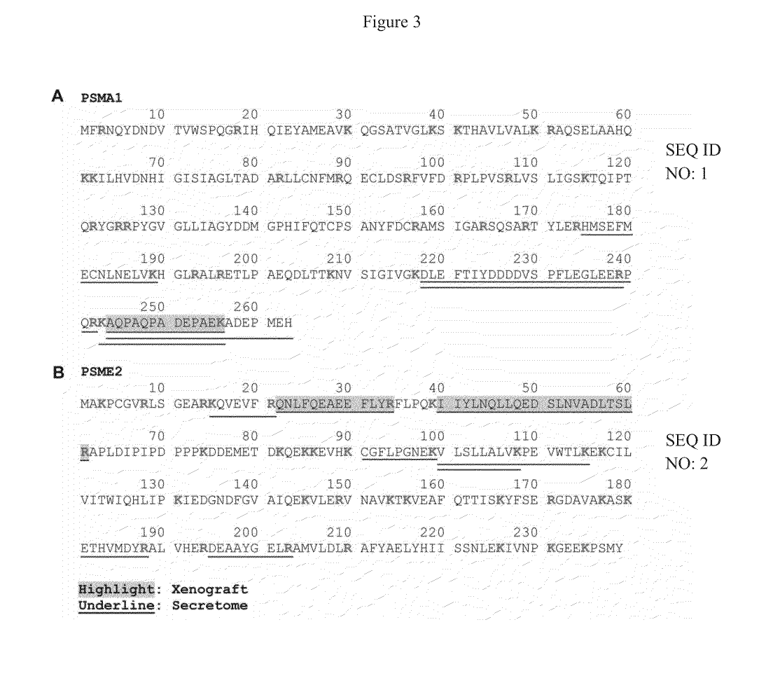

Examples of using the tumor secretome data to expand the utility of data from the xenograft plasma are shown in FIG. 3. PSMA1 (FIG. 3A) was identified by a total of 10 peptides in the mouse plasma, but only a single one of these was a uniquely human peptide. This protein could have been de-prioritized because of its high homology to a mouse counterpart and the possibility that the single uniquely human peptide in the plasma might have been a false positive identification or unknown mouse sequence variant. However, the tumor secretome identified an additional five peptides that were uniquely human, thus increasing the confidence of the species assignment for the original plasma identification. FIG. 3B shows PSME2, a protein identified by two uniquely human peptides in the plasma dataset, and therefore the species assignment as human is well supported. But, the secretome analysis identified six additional human peptides, thereby providing more proteotypic peptides for setting up MRM assays.

The secretome and plasma datasets were compared to a study by Pitteri et al. that identified candidate biomarkers by comparing a genetically engineered mouse model and secretomes of ovarian cancer cells.¹⁷ That study validated eight proteins found to be at higher abundance levels in ovarian cancer patients' plasma, and they also described identification of an additional nine proteins previously identified as ovarian cancer plasma biomarkers. Of the 17 candidate markers described by Pitteri et al., we identified eight proteins (CTSB, FASN, IGFBP2, LCN2, MIF, THBS1, WFDC2, and NRCAM) in our secretome analysis, and three proteins (FASN, IGFBP2, and LCN1) in the high-confidence xenograft plasma dataset. We also compared the results from the current study using OVCAR-3 cells to an earlier xenograft mouse study using an endometrioid EOC cell line (TOV-112D) where we identified three new biomarkers of ovarian cancer that could distinguish cancer patients from non-cancer patients.¹⁸ These three biomarkers, CLIC1, CTSD, and PRDX6, were all identified in the current study.

Overall, these results show that different biomarker discovery strategies result in detection of overlapping, but non-identical sets of biomarkers. These data also demonstrate that analysis of the tumor secretome in parallel with xenograft mouse plasma is useful for confirming candidate biomarkers detected in xenografted mouse plasma.

### Example 6

### Selection and Prioritization of Candidate Biomarkers

The human proteins identified in the secretome are far too numerous for verification and validation in patient samples using current methods. Although effective methods for triaging datasets of this type are not well defined, a small subset of candidates was selected to test whether candidate biomarkers identified in the secretome, but not the mouse plasma, have potential as ovarian cancer plasma biomarkers. Because most proteins shed specifically by the tumor are likely to be low abundance in human plasma, any human secretome proteins matching a compiled list of 168 known low-abundant plasma proteins (≦100 ng/mL, based on published reports³⁰⁻³¹) were included.

For prioritization of the xenograft plasma proteome, we started with the 268 human proteins identified by two or more peptides (FIG. 2A). This dataset was further refined by removing a few trypsin and keratin contaminants that were missed at the initial contaminant-removal step due to ambiguous protein descriptions or isoform differences. In addition, known medium- to high-abundant plasma proteins (>100 ng/mL³⁰⁻³¹) and hemoglobins were removed because the contribution of a small tumor is unlikely to be discernible from the baseline level in the normal population.

### Example 7

### Verification of Candidate Biomarkers in the 15-50 kDa Region Using Label-Free Discovery Proteomics Analysis of Patient Pools

The candidate biomarkers selected from the xenograft plasma and tumor secretome analyses were cross-referenced against proteins identified in a label-free comparison of patient sera using a 4 h gradient. Benign samples were combined in a single pool (pool B, n=9), and advanced cancer specimens were combined in three pools (pool C1: stage 3, n=9; pool C2: stage 3, n=9; pool C3: stage 4, n=5) as described in FIG. 8. Acquisition of full MS and data-dependent MS/MS scans were identical to those described for the xenograft proteome analyses, with the exception that ions subjected to MS/MS were excluded from repeated analysis for 180 s.

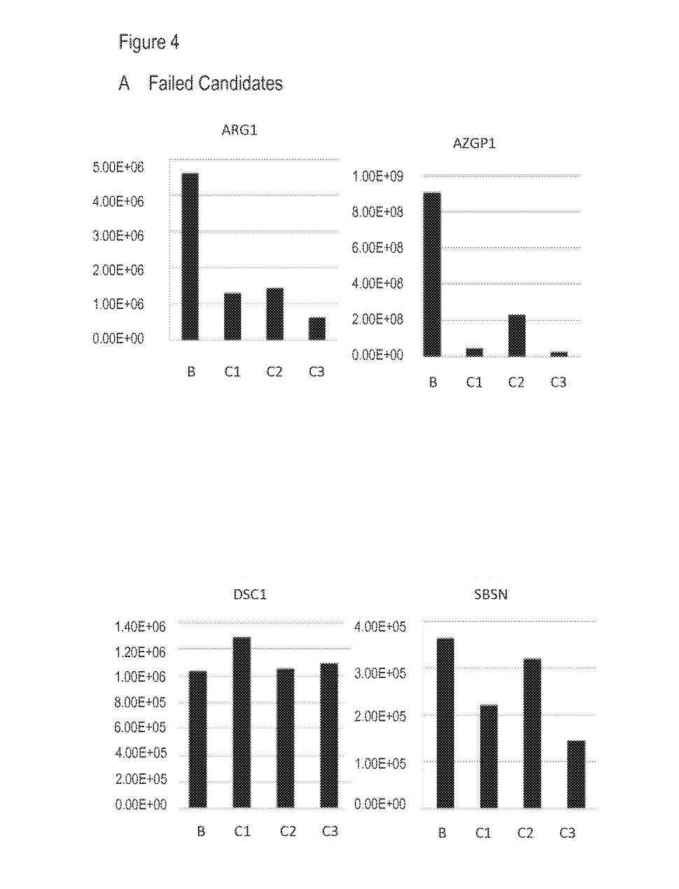

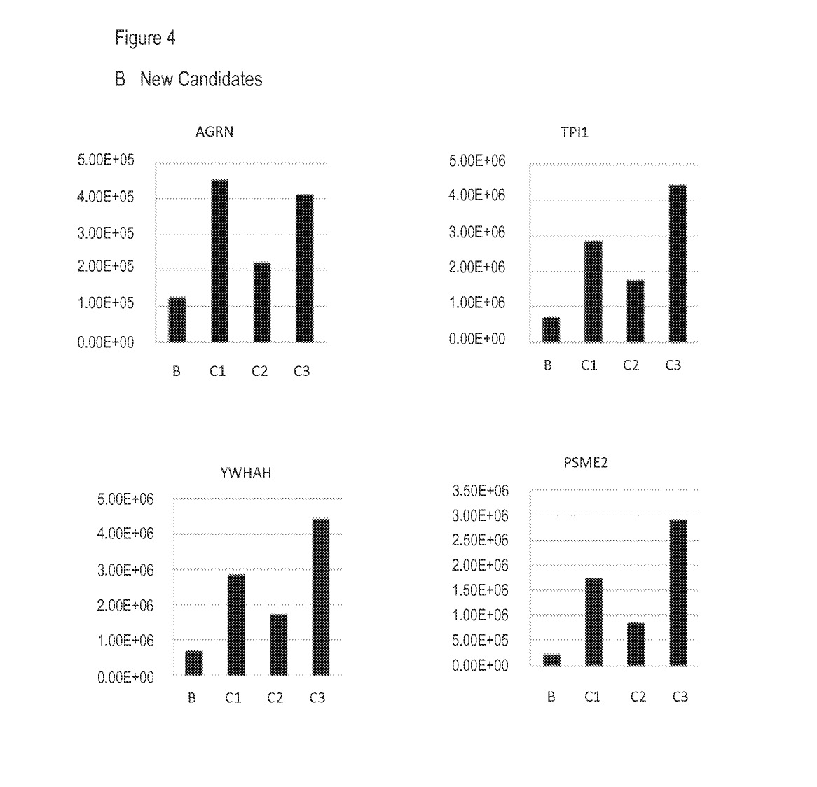

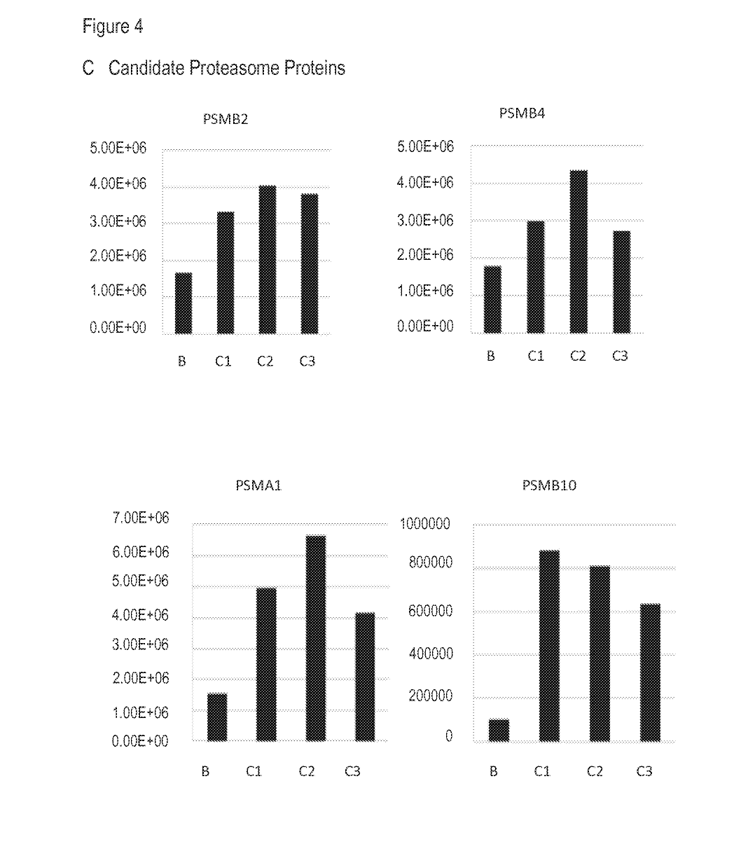

Proteins were initially compared across pools using spectral counts. Selected proteins were further analyzed by comparing peptide ion signal intensities from the peptide report results using Elucidator software. Peptides were grouped into consensus proteins by protein description and peptide intensities were summed for each protein. The criteria for selecting candidates for further validation were proteins that showed increases in all three cancer pools compared with the benign serum, and where the average intensity of the three cancer pools was at least 1.5 times that of the benign serum pool. Candidates whose protein intensities did not increase in cancer were not considered to be good markers (FIG. 4A). ARG1 and AZGP failed because they showed decreases in cancer relative to benign disease—a trend that does not correlate with tumor burden. DSC1 and SPSN2 were not considered for further validation because the benign and cancer pools exhibited similar levels.

Approximately half of the high-priority candidates from the xenograft secretome and plasma that were expected to be in the 15-50 kDa range of the gel were detected in the patient pools, and nearly 50% of the detected candidate biomarkers met the criteria for an increase in cancer described above. FIG. 4B shows protein quantities across patient pools for representative promising candidates. One candidate, AGRN, is a 215 kDa protein previously identified as being upregulated in ovarian cancer tissue samples compared with normal and non-ovarian tissue samples,²⁸ but has not previously been reported to be a serum biomarker for EOC. In this study, it was identified by SDS-PAGE as both the intact protein and as a fragment in the tumor secretome. In contrast, only the lower molecular weight fragment was detected in the xenograft plasma. The peptides quantitated in FIG. 4 belong to the fragment and correlate with ovarian cancer at this level. We also identified multiple proteasome subunits that showed increases in ovarian cancer. The proteasome complex is responsible for degradation of proteins crucial to cell cycle regulation and apoptosis and has been recognized as a potential target for cancer therapy.³² Specific proteasome subunits, including PSMB2 and PSMB4, have been identified as upregulated in gene expression profiles of ovarian carcinomas.³³⁻³⁴ FIG. 4C shows the label-free quantitation of these two proteasome proteins, plus PSMA1 and PSMB10, which also display consistent increases in the cancer pools relative to the benign. Interestingly, circulating intact proteasomes have recently been reported to correlate with EOC,³⁵ but the assay used in that study did not distinguish specific isoforms or quantify subunits that may not have been in intact proteasomes.

In addition to verifying high-priority xenograft candidate biomarkers, the label-free comparison of cancer patient and benign pool dataset was used to conduct an independent unbiased search for candidates unique to this approach. In this case, the primary criteria were zero spectral counts in the benign pools with two or more spectral counts in at least one cancer pool. Candidates that met these criteria were then analyzed using the MS intensity data from Elucidator. Proteins identified by this method and that met the criteria for inclusion in further validation analyses included one known ovarian cancer biomarker, WFDC2 (HE4), and eight novel markers, four of which were identified in the secretome but did not cross-correlate with gene expression and, hence, were not among the high-priority candidates. None of the proteins identified by this method were identified in the xenograft plasma.

### Example 8

### MRM Assay Development

A total of 25 candidates exhibited elevated levels in cancer compared with benign patient sera in the label-free comparison and were selected for development of MRM assays. Robust MRM assays with at least two or three unique verified targeted peptides per protein were established for 21 of these 25 candidates. The only candidates where MRM assay development failed were four of eight candidates that were only identified from the label-free comparisons of the patient cancer and benign pools. These proteins were identified by a small number of peptides in the 4 h LC-MS/MS analyses and were apparently below the detection limit for the MRM assay, which used a shorter gradient. The 21 candidates where MRM assays were successfully set up and integrated into a single multiplexed assay are summarized in Table 2.

TABLE 2
Biomarkers for validation in patient serum pools.

| | | | |
|---|---|---|---|
| | | # Peptides^{a} | # Peptides^{a} |
| Gene | Protein Description | (Secretome) | (Plasma) |

Previously reported biomarkers:

| | | | |
|---|---|---|---|
| IGFBP2 | Insulin-like growth factor-binding protein 2^{b 36} | 9/5 | 5/4 |
| WFDC2 | WAP four-disulfide core domain protein 2^{c 37} | 2/0 | 0 |
| LRG1 | Leucine-rich alpha-2-glycoprotein^{b 38} | 0 | 1/1 |

Novel candidates identified from xenograft mouse plasma and verified in tumor
secretome

| | | | |
|---|---|---|---|
| AGRN | Agrin and agrin fragments | 61/9 | 5/1 |
| PSME2 | Proteasome activator complex subunit 2 | 8/11 | 2/8 |
| TPI1 | Triosephosphate isomerase | 7/18 | 4/10 |
| DDAH2 | N(G),N(G)-dimethylarginine | 7/8 | 1/4 |
| | dimethylaminohydrolase 2 | | |
| GM2A | GM2 ganglioside activator protein (GM2A) | 3/0 | 3/0 |
| YWHAB | 14-3-3 protein beta/alpha | 2/8 | 1/12 |
| YWHAH | 14-3-3 protein eta | 2/9 | 1/7 |
| PSMA1 | Proteasome subunit alpha type-1 | 6/14 | 1/10 |
| PSMB1 | Proteasome subunit beta type-1 | 3/9 | 1/6 |
| PSMB2 | Proteasome subunit beta type-2 | 1/10 | 1/6 |
| PSMB4 | Proteasome subunit beta type-4 | 3/5 | 2/5 |

Novel candidates identified from xenograft mouse tumor secretome

| | | | |
|---|---|---|---|
| FTH1 | Ferritin heavy chain | 8/2 | 0 |
| FTL | Ferritin light chain | 8/0 | 0 |
| TIMP2 | Metalloproteinase inhibitor 2 | 1/2 | 0 |

Novel candidates identified directly from label-free quantitation of patient sera

| | | | |
|---|---|---|---|
| CA13 | Carbonic anhydrase 13 | 7/0 | 0 |
| PSMB10 | Proteasome subunit beta type-10 | 6/3 | 0 |
| FGL2 | Fibroleukin | 0 | 0 |
| PGLYRP1 | Peptidoglycan recognition protein 1 | 0 | 0 |

^{a}Number of peptides that are uniquely human/number of peptides common to mouse and human homolog.
^{b}These previously known biomarkers were identified in the mouse plasma in this study.
^{c}This previously known biomarker was identified in the mouse secretome and the label-free quantitation of patient sera pools in this study.

Three proteins, IGFBP2, WFDC2, and LRG1 were included in this panel as references because they were previously reported by others as serum proteins associated with ovarian cancer.³⁶⁻³⁸ It should be noted that IGFBP2 has a plasma concentration of ˜300 ng/mL³⁰ and therefore does not meet our criteria of a low-abundant plasma protein in normal donors. However, because of its previous identification as a prognostic marker for ovarian cancer, we included it in our validation set.

### Example 9

### Potential Correlation of Biomarkers with Gene Expression

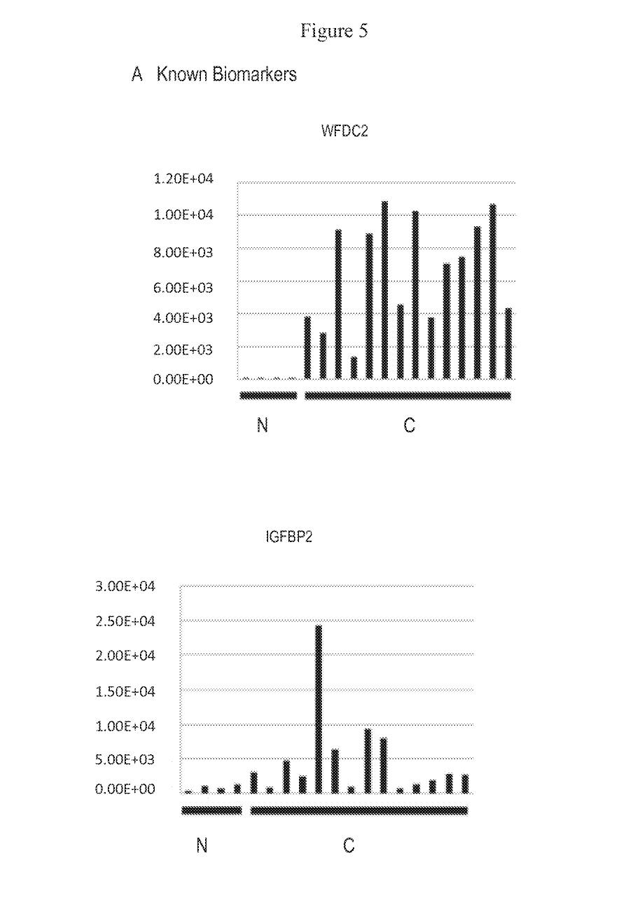

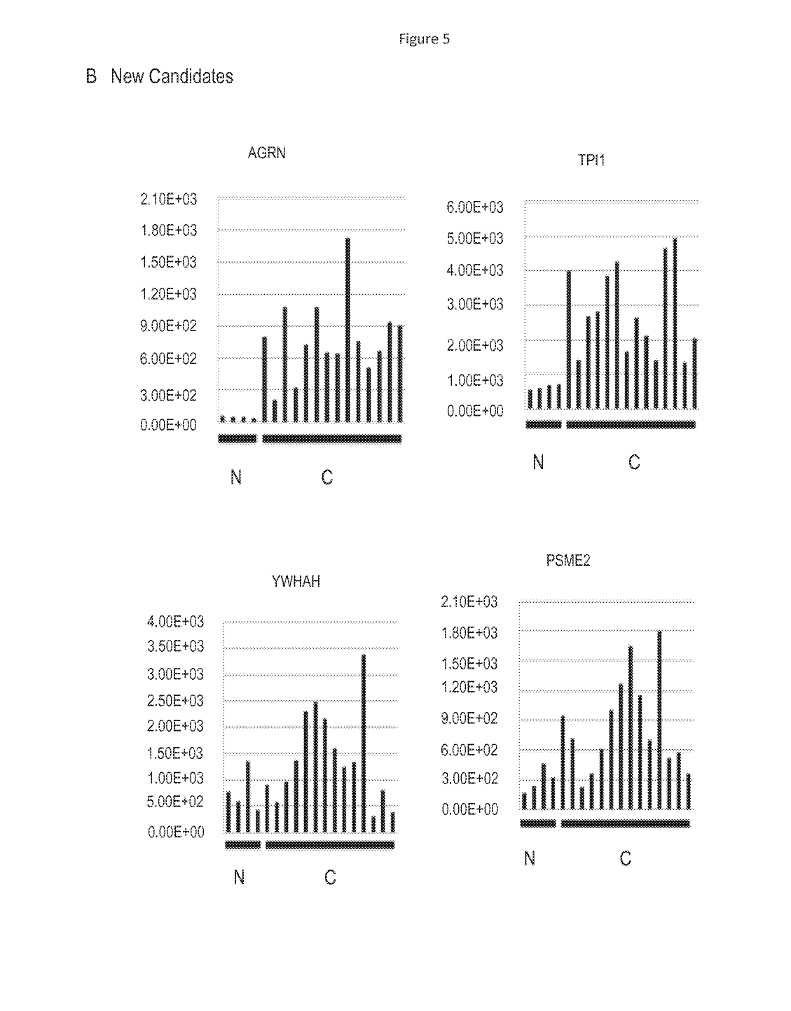

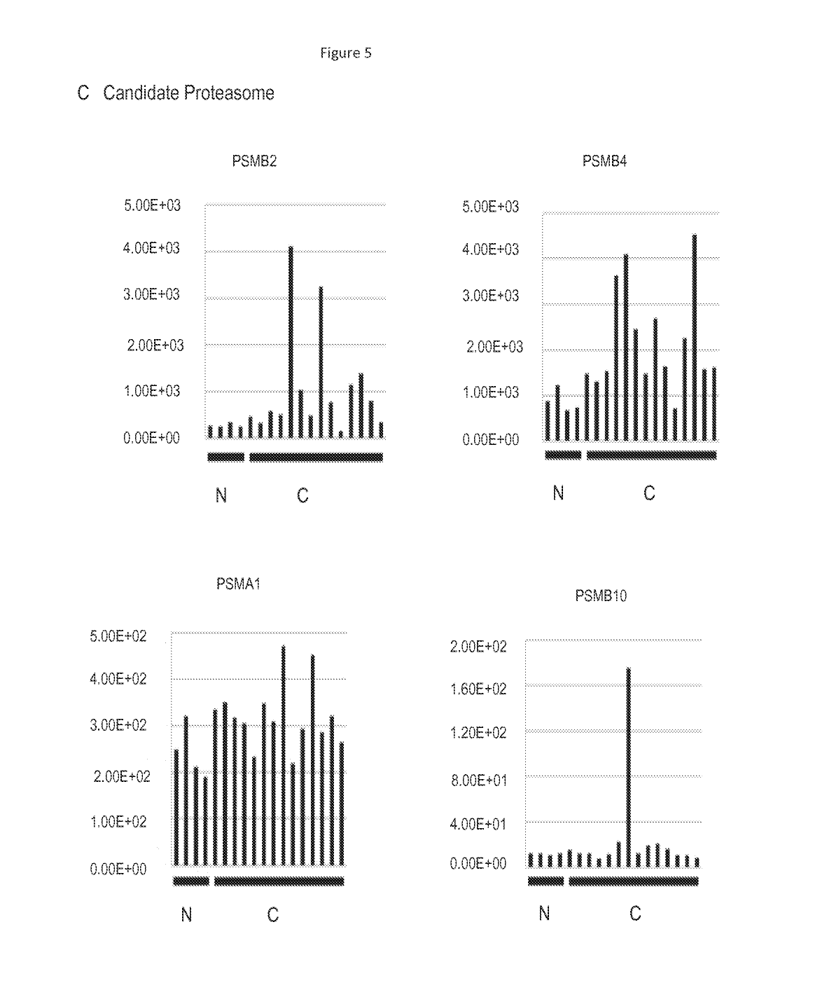

To further test the hypothesis that gene expression may be a useful indicator of whether a protein is likely to be a good serum biomarker, we queried our candidate biomarkers from Table 1 against published microarray hybridization data using BioGPS, a centralized gene portal of combined gene annotation resources.³⁹ Specifically, gene expression levels for normal ovarian tissue (n=4) and papillary serous ovarian carcinoma primary tumor samples (n=14)^{29, 40} were extracted for each of the candidate markers listed in Table 1. FIG. 5 shows examples of gene expression patterns for known ovarian cancer biomarkers (FIG. 5A) as well as novel candidates (FIGS. 5B and 5C), which include several candidate proteasome subunits. As shown in FIG. 5B, there is good correlation of gene expression with initial verification data for patient pools (FIG. 4B) for AGRN and TPI1. In contrast, the differences between normal and cancer tissue at the gene expression level is less well defined for YWAH and PSME2. Yet at the serum level these proteins show similar patterns to those for AGRN and TPI1.

Interestingly, gene expression varies widely across ovarian tissue specimens for the different proteasome subunits (FIG. 5C), but all four subunits show similar serum abundance level trends (FIG. 4C). These comparisons suggest that gene expression levels in normal and malignant ovarian tissues are not consistently good predictors of the blood levels of these proteins, although for specific proteins such a correlation may exist. This is not surprising because: gene expression levels do not always correlate with protein abundance within cells; shedding of proteins into the extracellular space and, more specifically, into the vascular system, does not necessarily depend upon the tissue levels of that protein; and changes in proteolytic processing or other processing of proteins that affect their blood concentration may differ between normal and cancer states.

### Example 10

### Preliminary MRM Validation of Biomarker Candidates in Patient Serum Pools

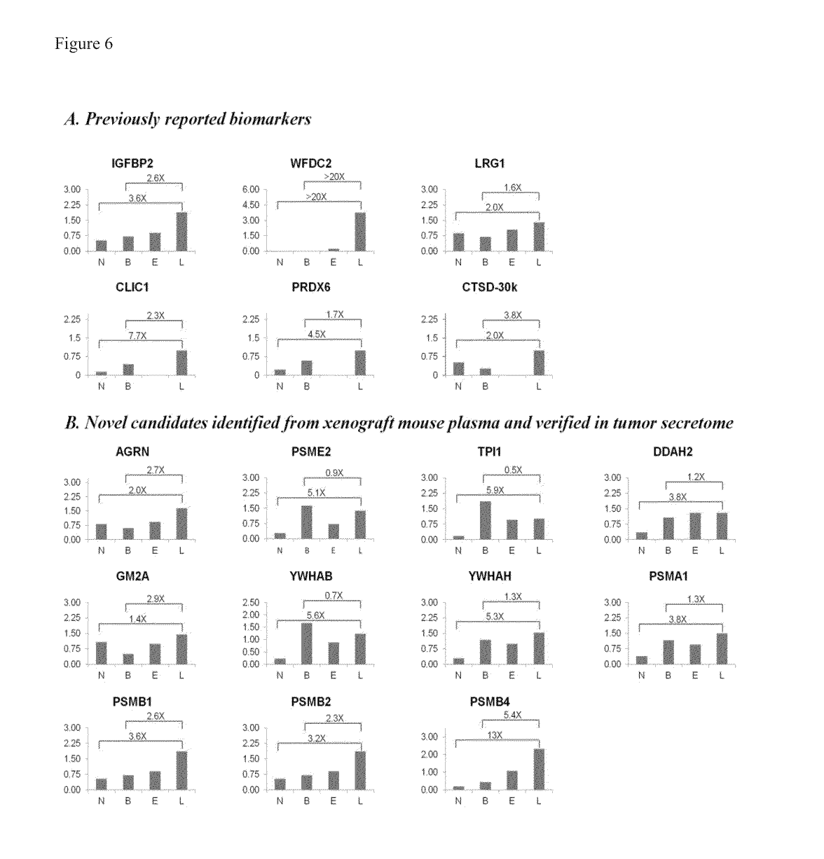

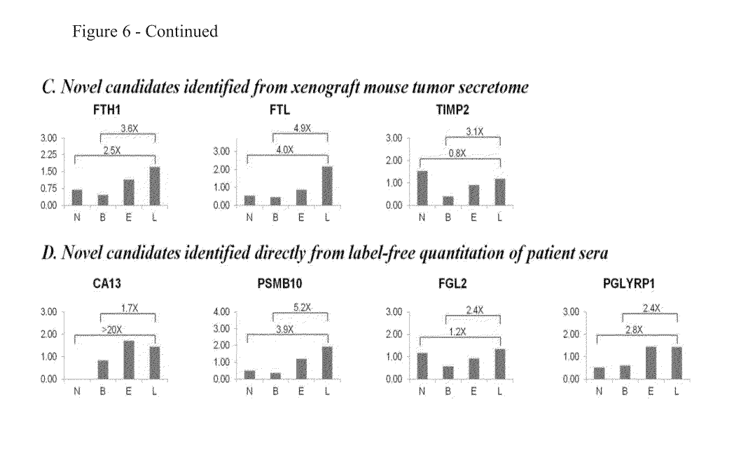

For pilot validation of the candidate biomarkers in Table 1, a normal serum pool (pool N; n=9), a benign ovarian tumor pool (pool B, n=10), an early-stage ovarian cancer pool (pool E: stage 1 and 2, n=18), and a late-stage cancer pool (pool L: stage 3, n=29) were prepared. Details for the patient samples used to prepare these pools are summarized FIG. 8. The peptides and transitions used in the integrated multiplex MRM assay, as well as the resulting relative quantitative data for the four pools, are shown in FIG. 7 and in Table S3 of the Supplemental materials of Beer et al, Beer et al, Identification of Multiple Novel Protein Biomarkers Shed by Human Serous Ovarian Tumors into the Blood of Immunocompromised Mice and Verified in Patent Sera, PLoS One, 8(3):e60129 (March 2013). Resulting relative protein quantities for the four pools are summarized in FIG. 6 for the 21 candidates.

A number of interesting conclusions can be drawn from these preliminary validation data by comparing the results for the novel biomarkers to those we observed for three biomarkers previously reported by others (IGFBP2, WFDC2, and LRG1) and three biomarkers previously reported by us (CLIC1, PRDX6, and CTSD-30K) as shown in FIG. 6A. First, most new biomarkers show fold increases in late-stage cancer similar to or larger than the increases observed for two of the previously known biomarkers—specifically IGFBP2 and LRG1. Also, all biomarkers show at least a two-fold increase in late-stage ovarian cancer relative to either the benign or normal pool or both. This suggests that most of the new biomarkers are likely to be as useful as these previously reported biomarkers. Second, protein abundance patterns across pools differ substantially for different biomarkers. Many of the biomarkers show the progressive increase in abundance, typical of many cancer biomarkers, as one goes from normal to benign to early-stage ovarian cancer to late-stage ovarian cancer. However, there are a number of exceptions where benign samples are unusually high and these proteins may be useful biomarkers for distinguishing benign disease from cancers. Third, most proteasome biomarkers show similar serum abundant level patterns with PSMB4 showing the largest fold increase between benign or normal vs. late-stage cancer, suggesting this may be the best biomarker within this group and that quantifying levels of individual subunits may be superior to the previously reported quantitation of intact circulating proteasomes.³⁵ Fourth, while the majority of biomarkers in this analysis were initially identified in xenograft mouse plasma, the additional biomarkers initially identified in the tumor secretome (FIG. 6C), and the additional biomarkers only identified in the label-free comparison of patient serum pools, exhibit abundance patterns and fold changes similar to the known biomarkers and the candidates from the xenograft plasma. This indicates that promising candidate biomarkers can be discovered by each of the three discovery approaches and there is substantial value in using all three methods in concert. This also suggests that the latter two proteomes could be further mined for additional biomarker candidates.

Each of the documents cited herein, and priority provisional patent application No. 61/720,616, are incorporated herein by reference. In addition, Beer et al, Identification of Multiple Novel Protein Biomarkers Shed by Human Serous Ovarian Tumors into the Blood of Immunocompromised Mice and Verified in Patent Sera, PLoS One, 8(3):e60129 (March 2013) is specifically incorporated by reference herein.

### REFERENCES

1. Jemal, A et al, Cancer statistics, 2008. CA Cancer J Clin 2008, 58, (2), 71-96.
2. Clarke-Pearson, D. L., Clinical practice. Screening for ovarian cancer. N Engl J Med 2009, 361, (2), 170-7.
3. Cannistra, S. A., Cancer of the ovary. N Engl J Med 2004, 351, (24), 2519-29.
4. Jacobs, I.; Bast, R. C., The CA125 tumour-associated antigen: a review of the literature. Hum Reprod 1989, 4, 1-12.
5. Woolas, R. P. et al, Elevation of multiple serum markers in patients with stage I ovarian cancer. J Natl Cancer Inst 1993, 85, (21), 1748-51.
6. Bast, R. C. et al, A radioimmunoassay using a monoclonal antibody to monitor the course of epithelial ovarian cancer. N Engl J Med 1983, 309, (15), 883-7.
7. Sasaroli, D. et al, Beyond CA125: the coming of age of ovarian cancer biomarkers. Are we there yet? Biomark Med 2009, 3, (3), 275-288.
8. Cramer, D. W. et al, Ovarian cancer biomarker performance in prostate, lung, colorectal, and ovarian cancer screening trial specimens. Cancer Prev Res (Phila) 2011, 4, (3), 365-74.
9. Zhu, C. S. et al, A framework for evaluating biomarkers for early detection: validation of biomarker panels for ovarian cancer. Cancer Prev Res (Phila) 2011, 4, (3), 375-83.
10. Hoffman, S. A. et al, Higher dimensional (Hi-D) separation strategies dramatically improve the potential for cancer biomarker detection in serum and plasma. J Chromatogr B Analyt Technol Biomed Life Sci 2007, 849, (1-2), 43-52.
11. Gortzak-Uzan, L. et al, A proteome resource of ovarian cancer ascites: integrated proteomic and bioinformatic analyses to identify putative biomarkers. J Proteome Res 2008, 7, (1), 339-51.
12. Faca, V. M. et al, Proteomic analysis of ovarian cancer cells reveals dynamic processes of protein secretion and shedding of extra-cellular domains. PLoS One 2008, 3, (6), e2425.
13. Kuk, C et al, Mining the ovarian cancer ascites proteome for potential ovarian cancer biomarkers. Mol Cell Proteomics 2009, 8, (4), 661-9.
14. Amon, L. M et al., Integrative proteomic analysis of serum and peritoneal fluids helps identify proteins that are up-regulated in serum of women with ovarian cancer. PLoS One 2010, 5, (6), e11137.
15. Zhang, Y. et al, The ovarian cancer-derived secretory/releasing proteome: A repertoire of tumor markers. Proteomics 2012, 12, (11), 1883-91.
16. Faca, V. M.; Hanash, S. M., In-depth proteomics to define the cell surface and secretome of ovarian cancer cells and processes of protein shedding. Cancer Res 2009, 69, (3), 728-30.
17. Pitteri, S. J. et al, Integrated proteomic analysis of human cancer cells and plasma from tumor bearing mice for ovarian cancer biomarker discovery. PLoS One 2009, 4, (11), e7916.
18. Tang, H. Y. et al, A xenograft mouse model coupled with in-depth plasma proteome analysis facilitates identification of novel serum biomarkers for human ovarian cancer. J Proteome Res 2012, 11, (2), 678-91.
19. Beer, L. A. et al, Systematic discovery of ectopic pregnancy serum biomarkers using 3-D protein profiling coupled with label-free quantitation. J Proteome Res 2011, 10, (3), 1126-38.
20. Zuo, X. et al, Towards global analysis of mammalian proteomes using sample prefractionation prior to narrow pH range two-dimensional gels and using one-dimensional gels for insoluble and large proteins. Electrophoresis 2001, 22, (9), 1603-15.
21. Tang, H. Y.; Speicher, D. W., Complex proteome prefractionation using microscale solution isoelectrofocusing. Expert Rev Proteomics 2005, 2, (3), 295-306.
22. Tang, H. Y. et al, A novel four-dimensional strategy combining protein and peptide separation methods enables detection of low-abundance proteins in human plasma and serum proteomes. Proteomics 2005, 5, (13), 3329-42.
23. Wang, H. et al, Data analysis strategy for maximizing high-confidence protein identifications in complex proteomes such as human tumor secretomes and human serum. J Proteome Res 2011, 10, (11), 4993-5005.
24. Liu, Q. et al, The proteome of the mouse photoreceptor sensory cilium complex. Mol Cell Proteomics 2007, 6, (8), 1299-317.
25. MacLean, B et al, Skyline: an open source document editor for creating and analyzing targeted proteomics experiments. Bioinformatics 2010, 26, (7), 966-8.
26. Lu, K. H et al, Selection of potential markers for epithelial ovarian cancer with gene expression arrays and recursive descent partition analysis. Clin Cancer Res 2004, 10, (10), 3291-300.
27. Hibbs, K et al., Differential gene expression in ovarian carcinoma: identification of potential biomarkers. Am J Pathol 2004, 165, (2), 397-414.
28. Tchagang, A. B. et al, Early detection of ovarian cancer using group biomarkers. Mol Cancer Ther 2008, 7, (1), 27-37.
29. Welsh, J. B. et al, Analysis of gene expression profiles in normal and neoplastic ovarian tissue samples identifies candidate molecular markers of epithelial ovarian cancer. Proc Natl Acad Sci USA 2001, 98, (3), 1176-81.
30. Polanski, M.; Anderson, N. L., A list of candidate cancer biomarkers for targeted proteomics. Biomark Insights 2007, 1, 1-48.
31. Anderson, L., Candidate-based proteomics in the search for biomarkers of cardiovascular disease. J Physiol 2005, 563, (Pt 1), 23-60.
32. Voorhees, P. M. et al, The proteasome as a target for cancer therapy. Clin Cancer Res 2003, 9, (17), 6316-25.
33. Yue, W. et al Screening and identification of ovarian carcinomas related genes. Ai Zheng 2004, 23, (2), 141-5.
34. Nowee, M. E et al, DNA profiling of primary serous ovarian and fallopian tube carcinomas with array comparative genomic hybridization and multiplex ligation-dependent probe amplification. J Pathol 2007, 213, (1), 46-55.
35. Heubner, M et al., The prognostic impact of circulating proteasome concentrations in patients with epithelial ovarian cancer. Gynecol Oncol 2011, 120, (2), 233-8.
36. Baron-Hay, S et al., Elevated serum insulin-like growth factor binding protein-2 as a prognostic marker in patients with ovarian cancer. Clin Cancer Res 2004, 10, (5), 1796-806.
37. Hellstrom, I et al, The HE4 (WFDC2) protein is a biomarker for ovarian carcinoma. Cancer Res 2003, 63, (13), 3695-700.
38. Andersen, J. D et al, Leucine-rich alpha-2-glycoprotein-1 is upregulated in sera and tumors of ovarian cancer patients. J Ovarian Res 2010, 3, 21.
39. Wu, C et al, BioGPS: an extensible and customizable portal for querying and organizing gene annotation resources. Genome Biol 2009, 10, (11), R130.
40. Su, A. I et al, Molecular classification of human carcinomas by use of gene expression signatures. Cancer Res 2001, 61, (20), 7388-93.

1. A diagnostic reagent comprising at least one ligand capable of specifically complexing with, binding to, or quantitatively detecting or identifying a single target biomarker selected from the group consisting of:
a. Agrin and agrin fragments;
b. Proteasome activator complex subunit 2;
c. Triosephosphate isomerase;
d. N(G),N(G)-dimethylarginine dimethylaminohydrolase 2;
e. GM2 ganglioside activator protein (GM2A);
f. 14-3-3 protein beta/alpha;
g. 14-3-3 protein eta;
h. Proteasome subunit alpha type-1;
i. Proteasome subunit beta type-1;
j. Proteasome subunit beta type-2;
k. Proteasome subunit beta type-4;
l. Ferritin heavy chain;
m. Ferritin light chain;
n. Metalloproteinase inhibitor 2;
o. Carbonic anhydrase 13;
P. Proteasome subunit beta type-10;
q. Fibroleukin;
r. Peptidoglycan recognition protein 1; and
s. an isoform, pro-form, modified molecular form, or peptide fragment of any of biomarkers (a) through (r), proteins in the same biomarker family or expressed from a related gene, having at least 20% sequence homology or sequence identity with any biomarker (a) through (r);
wherein at least one ligand is associated with a detectable label or with a substrate.

2. The reagent according to claim 1, comprising multiple ligands selected from (a) through (s), each ligand directed to a different biomarker.

3. The reagent according to claim 1, comprising one of the following combinations:
(i) one or more of ligands (a), (f), (g), (i), (j), (k), (m), (o), (p) and (r); or
(ii) one or more of ligands (b), (c), (d), (e), (h), (n), and (q).

4. The reagent according to claim 1, further comprising at least one ligand that specifically complexes with, binds to, quantitatively detects or identifies at least one additional biomarker.

5. The reagent according to claim 4, wherein the additional biomarker is selected from CA125, CLIC1, PRDX6, CTSD-30K, IGFBP2, WFDC2 (HE4), LRG1, and an isoform, pro-form, modified molecular form, or peptide fragment thereof.

6. The reagent according to claim 1, wherein each said ligand is selected from an antibody or fragment of an antibody, antibody mimic or equivalent that binds to or complexes with a biomarker of (a) through (s).

7. The reagent according to claim 1, wherein said substrate is a microarray, a microfluidics card, a chip, a bead, or a chamber.

8. A kit, panel or microarray comprising at least two diagnostic reagents of claim 1, each reagent identifying a different biomarker.

9. The kit, panel or microarray according to claim 8, comprising diagnostic reagents that bind to or complex individually with at least one additional known biomarker, isoform, pro-form, modified molecular form, or peptide fragment or homolog thereof.

10. The kit, panel or microarray according to claim 8, wherein the additional biomarker is selected from CA125, CLIC1, PRDX6, CTSD-30K, IGFBP2, WFDC2 (HE4) and LRG1.

11. The kit, panel or microarray according to claim 8, which comprises a substrate upon which said ligand is immobilized.

12. A method for diagnosing or detecting or monitoring the progress of ovarian cancer in a subject comprising:
(i) contacting a sample obtained from a test subject with the diagnostic reagent of claim 1;
(ii) detecting or measuring in the sample or from a protein level profile generated from the sample, the protein levels of one or more of the biomarkers (a) to (s), or ratios thereof;
(iii) comparing the protein levels of the biomarker in the subject's sample or from a protein level profile or ratio of multiple said biomarkers, with the level of the same biomarker or biomarkers in a reference standard;
wherein a significant change in protein level of the subject's sample biomarker or biomarkers from that in the reference standard indicates a diagnosis, risk, or the status of progression or remission of ovarian cancer in the subject.

13. The method according to claim 12, wherein the reference standard is a mean, an average, a numerical mean or range of numerical means, a numerical pattern, a ratio, a graphical pattern or a protein level profile derived from the same biomarker or biomarkers in a reference subject or reference population.

14. The method according to claim 12, wherein the reference standard is selected from a reference subject or reference population selected from the group consisting of
(I) a reference human subject or a population of subjects having no ovarian cancer;
(II) a reference human subject or a population of subjects having benign ovarian nodules;
(III) a reference human subject or a population of subjects following surgical removal of an ovarian tumor;
(IV) a reference human subject or a population of subjects prior to surgical removal of an ovarian tumor;
(V) a reference human subject or a population of subjects following therapeutic treatment for an ovarian tumor;
(VI) a reference human subject or a population of subjects prior to therapeutic treatment for an ovarian tumor;
(VII) a reference human subject or a population of subjects without ovarian cancer but which tests positive for a protein level of CA125;
(VIII) a reference human subject or a population of subjects with ovarian cancer but which tests negative for a protein level of CA125;
(IX) the same subject who provided a temporally earlier biological sample;
(X) a reference human subject or a population of subjects having early stage ovarian cancer;
(XI) a reference human subject or a population of subjects having advanced stage ovarian cancer;
(XII) a reference human subject or a population of subjects having a subtype of epithelial ovarian cancer;
(XIII) a reference human subject or a population of subjects having serous or serous papillary ovarian cancer;
(XIV) a reference human subject or a population of subjects having mucinous ovarian cancer;
(XV) a reference human subject or a population of subjects having clear cell ovarian cancer;
(XVI) a reference human subject or a population of subjects having endometrioid ovarian cancer;
(XVII) a reference human subject or a population of subjects having Mullerian ovarian cancer;
(XVIII) a reference human subject or a population of subjects having undifferentiated ovarian cancer;
(XIX) a reference human subject or a population of subjects having serous papillary adenocarcinoma; and
(XX) a reference human subject or a population of subjects having sarcoma.

15. The method according to claim 12, wherein the subject's sample has been provided at a time selected from the group consisting of:
(A) before any ovarian cancer diagnosis;
(B) after a diagnosis of ovarian cancer;
(C) following surgical removal of an ovarian tumor;
(D) prior to surgical removal of an ovarian tumor;
(E) periodically following therapeutic treatment for an ovarian tumor;
(F) periodically during therapeutic treatment for an ovarian cancer;
(G) following tumor reoccurrence and during treatment or monitoring of the reoccurring tumor
(H) prior to therapeutic treatment for an ovarian tumor; and
(I) before diagnosis but with clinical symptoms of abdominal paid or abdominal symptom of unknown origin.

16. The method according to claim 12, wherein said change in protein level of each said biomarker comprises an increase in comparison to said reference or control.

17. The method according to claim 12, wherein said diagnosis comprises: early diagnosis of disease, monitoring relapse after initial diagnosis and treatment, predicting clinical outcome, or determining the best clinical treatment.

18. The method according to claim 12, wherein the biological sample is selected from group consisting of whole blood, plasma, serum, circulating tumor cells, ascites fluid, tumor secretome fluid, peritoneal fluid and tumor tissue.

19. The method according to claim 12, comprising performing a serum or plasma sandwich ELISA or a mass spectrometry-based test.

20. The method according claim 12, wherein the biomarker or biomarkers are present in different levels or abundance profiles in biological samples of two or more of the conditions selected from:
(1) no ovarian cancer;
(2) benign ovarian nodules;
(3) a subtype of epithelial ovarian cancer
(4) following surgical removal of an ovarian tumor;
(5) prior to surgical removal of an ovarian tumor;
(6) following therapeutic treatment for an ovarian tumor;
(7) periodically during treatment for ovarian tumor;
(8) prior to therapeutic treatment for an ovarian tumor;
(9) undiagnosed clinical symptoms of abdominal pain or other abdominal condition of unknown origin;
(10) early stage ovarian cancer;
(11) advanced stage ovarian cancer;
(12) ovarian sarcoma,
(13) serous ovarian cancer;
(14) mucinous ovarian cancer;
(15) clear cell ovarian cancer;
(16) endometrioid ovarian cancer;
(17) Mullerian ovarian cancer;
(18) undifferentiated ovarian cancer; and
(19) serous papillary adenocarcinoma.
